(19) United States
(12) Patent Application Publication
(10) Pub. No.: US 2021/0085662 A1
(43) Pub. Date: Mar. 25, 2021

(54) METHODS AND PHARMACEUTICAL COMPOSITIONS FOR REDUCING PERSISTENCE AND EXPRESSION OF EPISOMAL VIRUSES

(71) Applicants: INSERM (Institut National de la Santé et de la Recherche Médicale), Paris (FR); Centre National de la Recherche Scienfique (CNRS), Paris (FR); Ecole Normale Superieure de Lyon, Lyon (FR); Université Claude Bernard Lyon 1, Villeurbanne (FR)

(72) Inventors: Vincent LOTTEAU, Lyon (FR); Patrice ANDRE, Lyon (FR)

(21) Appl. No.: 16/499,026

(22) PCT Filed: Mar. 29, 2018
(86) PCT No.: PCT/EP2018/058124

§ 371 Date: Sep. 27, 2019

(30) Foreign Application Priority Data
Mar. 30, 2017 (EP) 17305375.2

(51) Int. Cl. A61K 31/4439; A61K 31/42; A61K 31/575; A61K 31/496; A61K 31/5517; A61K 31/444

(52) U.S. Cl. CPC A61K 31/4439; A61K 31/42; A61K 31/575; A61K 45/06; A61K 31/5517; A61K 31/444; A61K 31/496

(57) ABSTRACT

The inventors surprisingly found that FXR plays a determinant role in the maintenance of viral episome in cells from tissues that are not specialized in bile salt synthesis and transport as the liver or the intestine. In particular, the inventors show that FXR agonist could be suitable for inhibiting the replication of viruses (e.g. BKV and HIV-1) that persist in the cell in an episomal and extrachromosomal form of DNA. Accordingly the present invention relates to a method of reducing persistence and expression of an episomal virus in a subject in need thereof comprising administrating to the subject a therapeutically effective amount of a FXR agonist.

### FIELD OF THE INVENTION

The present invention relates to methods and pharmaceutical compositions for reducing persistence and reduction of episomal viruses in subject in need thereof.

### BACKGROUND OF THE INVENTION

The replication intermediate of numerous DNA viruses are organized in a chromatin-like structure during their life cycle which is often referred as episome. For instance and typically, the circular genomes of papovaviruses, Simian virus 40 (SV40), and polyoma virus exist as minichromosomes composed of cellular histones organized in nucleosomes. Other viruses, such as the latent genomes of alpha herpes viruses, such as herpes simplex virus type 1, and of gammaherpesviruses, such as Epstein-Barr virus and Kaposi's sarcoma-associated herpesvirus, are maintained as episomal chromatin. Previous works found that patients undergoing highly active antiretroviral therapy (HAART) exhibited increased levels of unintegrated, episomal HIV-1 DNA, suggestive of de novo infection (Buz& MJ, Massanella M, Llibre J M, et al. HIV-1 replication and immune dynamics are affected by raltegravir intensification of HAART-suppressed subjects. Nat. Med. 2010; 16(4):460-465). It was also shown that certain tissues, such as lymph nodes and gut-associated lymphatic tissue, harbor increased levels of episomal viral DNA, suggesting that these tissues are potential sites of de novo replication. Accordingly, there is an unmet need for methods and pharmaceutical composition of eradicating persistence of episomal viruses.

Recent studies show that FXR agonists could be suitable for the treatment of hepatitis B virus (HBV) infection (WO 2015036442) and suggests that HBV replication and altered liver bile salt metabolism homeostasis and FXR regulation seem to be interdependent, and might contribute to the persistence of HBV infection. However the role of FXR in the persistence of episomal viruses has not yet been investigated.

### SUMMARY OF THE INVENTION

The present invention relates to methods and pharmaceutical compositions for reducing persistence and expression of episomal viruses in subject in need thereof. In particular, the present invention is defined by the claims.

### DETAILED DESCRIPTION OF THE INVENTION

The inventors surprisingly found that FXR plays a determinant role in the maintenance of active viral episome in cells from tissues that are not specialized in bile salt synthesis and transport as the liver or the intestine. In particular, the inventors show that FXR agonist could be suitable for inhibiting the replication of viruses (e.g. BKV and HIV-1) that persist in the cell in an episomal and extrachromosomal form of DNA.

Accordingly the first object of the present invention relates to a method of reducing persistence and expression of an episomal virus in a subject in need thereof comprising administrating to the subject a therapeutically effective amount of a FXR agonist.

In some embodiments, the subject can be human or any other animal (e.g., birds and mammals) susceptible to infection with an episomal virus (e.g. domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a non-human animal. In some embodiments, the subject is a farm animal or pet. In some embodiments, the subject is a human. In some embodiments, the subject is a human infant. In some embodiments, the subject is a human child. In some embodiments, the subject is a human adult. In some embodiments, the subject is an elderly human. In some embodiments, the subject is a premature human infant.

As used herein the term “episomal virus” refers to a virus, which requires episomal replication to persist in the subject. Episomal replication means that the virus is able to replicate without integration into host's chromosomal DNA and without gradual loss from a dividing host cell also meaning that said virus replicates episomally. By extension, “episomal virus” refers also to a virus whose replication requires presence of nuclear extra-chromosomal forms of DNA at least at some step of its genome replication and transcription. For instance, for retrovirus, following infection, the linear double-stranded retroviral DNA (dsDNA) is generated by means of reverse transcription upon entry into the host cell. The retroviral dsDNA is then translocated into the nucleus as an extra-chromosomal DNA, which is a mandatory replication intermediate. There the viral dsDNA can be integrated in the cellular chromatin or circularized to form single or double LTR episome. The retrovirus carries ori and the host cell provides the cognate replication protein of small DNA virus, which is used for amplification and replication of the circular DNA retroviral genome.

Example of episomal viruses, which infect vertebrates include but are not limited to viruses belonging to Adenoviridae, Retroviridae, Herpesviridae, Papovaviridae (Polyomaviridae and Papillomaviridae) Parvoririnae and families.

In some embodiments, the episomal virus is an adenovirus. As used herein, the term “adenovirus” has its general meaning in the art and refers to a member of the family Adenoviridae, which are medium-sized (90-100 nm), nonenveloped (without an outer lipid bilayer) viruses with an icosahedral nucleocapsid containing a double stranded DNA genome. In particular, human adenoviruses include the A-F subgenera and the individual serotypes thereof. The A-F subgenera includes, but is not limited to, human adenovirus types 1, 2, 3, 4, 4a, 5, 6, 7, 8, 9, 10, 11 (Ad11A and Ad11P), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48 and 91.

In some embodiments, the episomal virus is a herpesvirus. As used herein, the term “Herpesvirus” has its general meaning in the art and refers to a member of the family Herpesviridae, The family name is derived from the Greek word herpein (“to creep”), referring to the latent, recurring infections typical of this group of viruses. Example of Herpesviruses include but are not limited to Iltovirus; Proboscivirus; Cytomegalovirus; Mardivirus; Rhadinovirus; Macavirus; Roseolovirus; Simplexvirus; Scutavirus; Varicellovirus; Percavirus; Lymphocryptovirus; Muromegalovirus. In particular, the method of the present invention is particularly suitable for eradicating persistence of Herpes simplex virus-1 (HSV-1), Herpes simplex virus-2 (HSV-2), Varicella zoster virus (VZV), Epstein-Barr virus (EBV), lymphocryptovirus, Cytomegalovirus (CMV), Roseolovirus, Herpes lymphotropic virus and Kaposi's sarcoma-associated herpesvirus.

In some embodiments, the episomal virus is a papillomavirus. As used herein, the term “papillomavirus” relates to a DNA virus from the Papillomaviridae family of viruses that infects the skin and mucous membranes of mammals. For human PV (HPV), more than 110 HPV genotypes have been described (de Villiers, E. M., C. Fauquet, T. R. Broker, H. U. Bernard, and H. zur Hausen. 2004. Classification of papillomaviruses. Virology 324:17-27). Approximately 50 HPV genotypes are known to infect the mucosa. These mucosal genotypes are classified into three different groups based on their epidemiological association with cancer: “low-risk” human papillomaviruses (LR-HPV), “high-risk” human papillomaviruses (HR-HPV) and “putative high-risk” human papillomaviruses (pHR-HPV). It is also known that HR-HPVs can cause vulvar, anal, vaginal, penile, and oropharyngeal cancer, as well as vaginal intraepithelial neoplasia, anal intraepithelial neoplasia, vulvar intraepithelial neoplasia, and penile intraepithelial neoplasia. Preferably, HPVs are mucosal HPVs; more preferably, HPVs of the current invention are High-risk HPV genotypes (HR-HPVs), which are the main cause for the development of cervical cancer, more preferably HPVs are HPV 31, 33, 35, 39, 45, 51, 52, 56, 58, 59, 68, 73 and 82, most preferably HPV16 or HPV18.

In some embodiments, the episomal virus is a polyomavirus. As used herein, the term “Polyomavirus” has its general meaning in the art and refers to a member of family Polyomaviridae, which is a family of viruses whose natural hosts are primarily mammals and birds. Nine polyomaviruses have been discovered in humans: JCV, BKV, KI virus and WU virus, Merkel cell polyomavirus (MCV), Trichodysplasia sinulosa-associated polyomavirus (TSV), HPyV6, HPyV7, and HPyV9. Among these human polyomaviruses, JCV, BKV, and MCV cause serious complications and diseases. In some embodiments, the method of the invention is particularly suitable for the eradicating persistence of BKV. As used herein, the term “BK virus” or ‘BKV” has its general meaning in the art and refers to the 4 BKV serotypes that are known (serotypes I-IV; e.g., Knowles et al, J. Med. Virol. 28: 118-123, 1989).

In some embodiments, the episomal virus is a parvovirus. As used herein, the term “parvovirus” refers to a virus which is a member of the family Parvoviridae, preferably from the subfamily Parvoririnae. Exemplary parvoviruses include, but are not limited to, feline panleukopenia virus, canine parvovirus type 2, human parvovirus B19, minute virus of mice, bovine parvovirus, canine parvovirus, chicken parvovirus and goose parvovirus

In some embodiments, the episomal virus is a retrovirus. As used herein, the term “Retrovirus” has its general meaning in the art and refers to a member of family Retroviridae which are single-stranded positive-sense RNA viruses with a DNA intermediate and targets a host cell. Examples of retroviruses include, but are not limited to, bovine lentiviruses (e.g., bovine immunodeficiency virus, Jembrana disease virus), equine lentiviruses (e.g., equine infectious anemia virus), feline lentiviruses (e.g., feline immunodeficiency virus), ovine/caprine lentivirus (e.g., caprine arthritis-encephalitis virus, ovine lentivirus, visna virus) and primate lentiviruses, such as, human immunodeficiency virus (HIV), including human immunodeficiency virus type 1 (HIV-1), human immunodeficiency virus type 2 (HIV-2), human immunodeficiency virus type 3 (HIV-3), simian AIDS retrovirus SRV-1, including human T-cell lymphotropic virus type 4 (HIV-4) and simian immunodeficiency virus (SIV), Rous sarcoma virus, avian leukosis virus, and avian myeloblastosis virus, Avian carcinoma Mill Hill virus 2, Avian myelocytomatosis virus 29, Avian sarcoma virus CT10, Fujinami sarcoma virus, UR2 sarcoma virus, Y73 sarcoma virus, Jaagsiekte sheep retrovirus, Langur virus, Mason-Pfizer monkey virus, Squirrel monkey retrovirus, mouse mammary tumour virus, murine leukemia virus, feline leukemia virus, feline sarcoma virus, Gibbon ape leukemia virus, Guinea pig type-C oncovirus, Porcine type-C oncovirus, Finkel-Biskis-Jinkins murine sarcoma virus, Gardner-Arnstein feline sarcoma virus, Hardy-Zuckerman feline sarcoma virus, Harvey murine sarcoma virus, Kirsten murine sarcoma virus, Moloney murine sarcoma virus, Snyder-Theilen feline sarcoma virus, Woolly monkey sarcoma virus, avian reticuloendotheliosis viruses, including, but not limited to, Chick syncytial virus, Reticuloendotheliosis virus, and Trager duck spleen necrosis virus, bovine leukemia virus and Human T-lymphotropic virus.

In some embodiments, the episomal virus of the present invention does not infect intestine or liver cells of the subject.

As used herein, the term “persistence” or “persist” refers to the ability of the episomal virus to be maintained in the subject. An implication from reducing persistence of the virus is that the immediate symptoms caused by the virus would also be eliminated, as well as certain events or conditions associated with viral infection.

As used herein, the term “expression” refers to the ability of the DNA viral genome to be transcribed into viral RNA, either messenger or pregenomic, and to lead to the synthesis of viral protein and production of infectious particles.

The method of the present invention is thus particularly suitable for the treatment of viral infections mediated by episomal virus as above described. In particular, the method of the present invention is particularly suitable for the treatment of active, latent or reactivated infections. As used herein, an “active infection” refers to replication of an episomal virus in a cell. “Reactivation of an episomal virus” refers to the development of an active infection in a subject having a latent infection. As used herein, a “latent infection” refers to an infection that is not active. A subject having or suspected of having a latent infection includes a subject who has been exposed to an episomal virus, and/or in whom the presence of an episomal viral DNA and/or anti-virus antibodies have been clinically detected.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The method of the invention may be carried out with any subject. The subject is preferably a mammal, more preferably a primate and more preferably still, a human. Subjects may be male or female and may be of any age, including prenatal (i.e., in utero), neonatal, infant, juvenile, adolescent, adult, and geriatric subjects. Thus, in some cases the subjects may be pregnant female subjects.

In some embodiments, the subject has or is suspected of having a latent infection. In some embodiments, the subject has been diagnosed with an active infection.

In some embodiments, the subject is immunocompromised. Immunocompromised individuals include but are not limited to AIDS patients; patients on chronic immunosuppressive treatment regimens, such as organ transplant patients; patients with cancer such as Hodgkin's disease or lymphoma; and patients with autoimmune conditions being treated with mycophenolate mofetil or a biologic such as natalizumab, rituximab, or efalizumab. Such autoimmune conditions include, but are not limited to multiple sclerosis (MS), rheumatoid arthritis (RA), and systemic lupus erythematosis (SLE). Elderly patients with weakened immune systems that have or are suspected of having a latent polyomavirus infection are also at risk of developing a disease associated with a polyomavirus.

In some embodiments, the subject has a cancer and is administered with a cytoablative therapy such as chemotherapy or radiotherapy. As used herein, the term “cancer” has its general meaning in the art and includes, but is not limited to, solid tumors and blood borne tumors. The term “cytoablative therapy” has its general meaning in the art and refers to therapy that induce cytoablative effects on rapidly-proliferating cells via several different mechanisms, ultimately leading to cell cycle arrest and/or cellular apoptosis. Typically cytoablative therapy includes chemotherapy and radiotherapy. As used herein, the term “radiotherapy” has its general meaning in the art and refers to the medical use of ionizing radiation, generally as part of cancer treatment to control or kill malignant cells. As used herein the term “chemotherapy” has its general meaning in the art and refers to the medical use of chemotherapeutic agents effective in inhibiting tumor growth.

In some embodiments, the subject is a transplant subject who is administered with an immunosuppressive agent. In some embodiments, the transplant subject has at least one transplanted organ selected from the group consisting of kidney, bone marrow, liver, lung, stomach, bone, testis, heart, pancreas and intestine. In some embodiments, for the subject in need of an immunosuppressive agent, the compounds described herein may be administered in combination (concurrently or sequentially) with the immunosuppressive agent. As used herein the term “immunosuppressive agent” refers to any agent that inhibits or prevents an activity of the immune system of the subject. Non-limiting examples of immunosuppressive agents include antibodies (e.g., fully human or humanized antibodies) that specifically bind to CD20, CD25 (e.g., basiliximab or daclizumab), or CD3 (e.g., muromonab); calcineurin inhibitors (e.g., ciclosporin, pimecrolimus, tacrolimus, sirolimus, and/or cyclosporine); interferons (e.g., interferon-β); steroids (e.g., any of the steroids known in the art or described herein); interleukin-1 receptor antagonists; myophenolate mofetil; Prograph®; azathioprine; methotrexate; and/or TNF-α binding proteins (e.g., antibodies and/or soluble TNF-α receptors, e.g., infliximab, etanercept, and/or adalimumab).

In some embodiments, the method of the present invention is particularly suitable for eradicating HIV reservoir after highly active antiretroviral treatment (HAART). As used herein, the term “reservoir,” refers to the latent but replication competent HIV-1 proviruses present in resting CD4+ T cells. As used herein, the term “HAART” has its general leaning in the art and refers to any highly active antiretroviral therapy and is more recently referred to as combination antiretroviral therapy, or “cART”, used interchangeably herein with “CART”. HAART and cART are also used herein interchangeably. HAART may refer to three or more antiretroviral drugs in combination, and usually comprises one protease inhibitor and two or three reverse transcriptase inhibitors.

As used herein, the term “FXR” has its general meaning in the art and refers to the farnesoid X receptor, which is a nuclear receptor that is activated by supraphysiological levels of farnesol (Forman et al., Cell, 1995, 81,687-693). FXR, is also known as NR1H4, retinoid X receptor-interacting protein 14 (RIP14) and bile acid receptor (BAR). Containing a conserved DNA-binding domain (DBD) and a C-terminal ligand-binding domain (LBD), FXR binds to and becomes activated by a variety of naturally occurring bile acids (BAs), including the primary bile acid chenodeoxycholic acid (CDCA) and its taurine and glycine conjugates (Makishima et al., 1999; Parks et al., 1999; Wang et al., 1999. The human polypeptide sequences for FXR are deposited in nucleotide and protein databases under accession numbers NM_005123, Q96RI1, NP_005114 AAM53551, AAM53550, AAK60271.

As used herein, the term “FXR agonist” has its general meaning in the art and refers in particular to compounds that function by targeting and selectively binding the farnesoid X receptor (FXR) and which activate FXR by at least 40% above background in the assay described in Maloney et al. (J. Med. Chem. 2000, 43:2971-2974). In some embodiments, the FXR agonist of the invention is a selective FXR agonist. As used herein, the term “selective FXR agonist” refers to an FXR agonist that exhibits no significant cross-reactivity to one or more, ideally substantially all, of a panel of nuclear receptors consisting of LXRα, LXRβ, PPARα, PPARγ, PPARδ, RXRα, RARγ, VDR, SXR, ERα, ERβ, GR, AR, MR and PR. Methods of determining significant cross-reactivity are described in J. Med. Chem. 2009, 52, 904-907.

FXR agonists are well known to the skilled person. For example the skilled person may easily identified FXR agonist from the following publications:
Adorini L, Pruzanski M, Shapiro D. Farnesoid X receptor targeting to treat nonalcoholic steatohepatitis. Drug Discov Today. 2012 Sep.; 17(17-18):988-97. doi: 10.1016/j.drudis.2012.05.012. Epub 2012 May 29. Review.
Akwabi-Ameyaw A, Bass J Y, Caldwell R D, Caravella J A, Chen L, Creech K L, Deaton D N, Madauss K P, Man H B, McFadyen R B, Miller A B, Navas F 3rd, Parks D J, Spearing P K, Todd D, Williams S P, Bruce Wisely G. FXR agonist activity of conformationally constrained analogs of GW 4064. Bioorg Med Chem Lett. 2009 Aug. 15; 19(16):4733-9. doi: 10.1016/j.bmcl.2009.06.062. Epub 2009 Jun. 21.
Akwabi-Ameyaw A, Bass J Y, Caldwell R D, Caravella J A, Chen L, Creech K L, Deaton D N, Jones S A, Kaldor I, Liu Y, Madauss K P, Man H B, McFadyen R B, Miller A B, Iii F N, Parks D J, Spearing P K, Todd D, Williams S P, Wisely G B. Conformationally constrained farnesoid X receptor (FXR) agonists: Naphthoic acid-based analogs of GW 4064. Bioorg Med Chem Lett. 2008 Aug. 1; 18(15):4339-43. doi: 10.1016/j.bmcl.2008.06.073. Epub 2008 Jun. 28.
Akwabi-Ameyaw A, Caravella J A, Chen L, Creech K L, Deaton D N, Madauss K P, Man H B, Miller A B, Navas F 3rd, Parks D J, Spearing P K, Todd D, Williams S P, Wisely G B. Conformationally constrained farnesoid X receptor (FXR) agonists: alternative replacements of the stilbene. Bioorg Med Chem Lett. 2011 Oct. 15; 21(20):6154-60. doi: 10.1016/j.bmcl.2011.08.034. Epub 2011 Aug. 11.
Baghdasaryan A, Claudel T, Gumhold J, Silbert D, Adorini L, Roda A, Vecchiotti S, Gonzalez F J, Schoonjans K, Strazzabosco M, Fickert P, Trauner M. Dual farnesoid X receptor/TGR5 agonist INT-767 reduces liver injury in the Mdr2−/− (Abcb4−/−) mouse cholangiopathy model by promoting biliary HCO⁻₃ output. Hepatology. 2011 October; 54(4):1303-12. doi: 10.1002/hep.24537.
Bass J Y, Caldwell R D, Caravella J A, Chen L, Creech K L, Deaton D N, Madauss K P, Man H B, McFadyen R B, Miller A B, Parks D J, Todd D, Williams S P, Wisely G B. Substituted isoxazole analogs of farnesoid X receptor (FXR) agonist GW4064. Bioorg Med Chem Lett. 2009 Jun. 1; 19(11):2969-73. doi: 10.1016/j.bmcl.2009.04.047. Epub 2009 Apr. 18.
Bass J Y, Caravella J A, Chen L, Creech K L, Deaton D N, Madauss K P, Marr H B, McFadyen R B, Miller A B, Mills W Y, Navas F 3rd, Parks D J, Smalley T L Jr, Spearing P K, Todd D, Williams S P, Wisely G B. Conformationally constrained farnesoid X receptor (FXR) agonists: heteroaryl replacements of the naphthalene. Bioorg Med Chem Lett. 2011 Feb. 15; 21(4):1206-13. doi: 10.1016/j.bmcl.2010.12.089. Epub 2010 Dec. 23.
Buijsman et al., Curr. Med. Chem. 2005, 12, 1017
Chiang P C, Thompson D C, Ghosh S, Heitmeier M R. A formulation-enabled preclinical efficacy assessment of a farnesoid X receptor agonist, GW4064, in hamsters and cynomolgus monkeys. J Pharm Sci. 2011 November; 100(11):4722-33. doi: 10.1002/jps.22664. Epub 2011 Jun. 9.
Crawley, Expert Opin. Ther. Pat. 2010, 20, 1047
Feng S, Yang M, Zhang Z, Wang Z, Hong D, Richter H, Benson G M, Bleicher K, Grether U, Martin R E, Plancher J M, Kuhn B, Rudolph M G, Chen L. Identification of an N-oxide pyridine GW4064 analog as a potent FXR agonist. Bioorg Med Chem Lett. 2009 May 1; 19(9):2595-8. doi: 10.1016/j.bmcl.2009.03.008. Epub 2009 Mar. 9.
Flatt B, Martin R, Wang T L, Mahaney P, Murphy B, Gu X H, Foster P, Li J, Pircher P, Petrowski M, Schulman I, Westin S, Wrobel J, Yan G, Bischoff E, Daige C, Mohan R. Discovery of XL335 (WAY-362450), a highly potent, selective, and orally active agonist of the farnesoid X receptor (FXR). J Med Chem. 2009 Feb. 26; 52(4):904-7. doi: 10.1021/jm8014124.
Ghebremariam Y T, Yamada K, Lee J C, Johnson C L, Atzler D, Anderssohn M, Agrawal R, Higgins J P, Patterson A J, Boger R H, Cooke J P. FXR agonist INT-747 upregulates DDAH expression and enhances insulin sensitivity in high-salt fed Dahl rats. PLoS One. 2013 Apr. 4; 8(4):e60653. doi: 10.1371/journal.pone.0060653. Print 2013.
Gioiello A, Macchiarulo A, Carotti A, Filipponi P, Costantino G, Rizzo G, Adorini L, Pellicciari R. Extending SAR of bile acids as FXR ligands: discovery of 23-N-(carbocinnamyloxy)-3α,7α-dihydroxy-6α-ethyl-24-nor-513-cholan-23-amine. Bioorg Med Chem. 2011 Apr. 15; 19(8):2650-8. doi: 10.1016/j.bmc.2011.03.004. Epub 2011 Mar. 10.
Hoekstra M, van der Sluis R J, Li Z, Oosterveer M H, Groen A K, Van Berkel T J. FXR agonist GW4064 increases plasma glucocorticoid levels in C57BL/6 mice. Mol Cell Endocrinol. 2012 Oct. 15; 362(1-2):69-75. doi: 10.1016/j.mce.2012.05.010. Epub 2012 May 27.
Iguchi Y, Kihira K, Nishimaki-Mogami T, Une M. Structure-activity relationship of bile alcohols as human farnesoid X receptor agonist. Steroids. 2010 January; 75(1):95-100. doi: 10.1016/j.steroids.2009.11.002. Epub 2009 Nov. 12.
Lin H R. Triterpenes from Alisma orientalis act as farnesoid X receptor agonists. Bioorg Med Chem Lett. 2012 Jul. 15; 22(14):4787-92. doi: 10.1016/j.bmcl.2012.05.057. Epub 2012 May 23.
Lefebvre P, Cariou B, Lien F, Kuipers F, Staels B. Role of bile acids and bile acid receptors in metabolic regulation. Physiol Rev. 2009 January; 89(1):147-91. doi: 10.1152/physrev.00010.2008.
Lundquist J T, Harnish D C, Kim C Y, Mehlmann J F, Unwalla R J, Phipps K M, Crawley M L, Commons T, Green D M, Xu W, Hum W T, Eta J E, Feingold I, Patel V, Evans M J, Lai K, Borges-Marcucci L, Mahaney P E, Wrobel J E. Improvement of physiochemical properties of the tetrahydroazepinoindole series of farnesoid X receptor (FXR) agonists: beneficial modulation of lipids in primates. J Med Chem. 2010 Feb. 25; 53(4):1774-87. doi: 10.1021/jm901650u.
Ma Y, Huang Y, Yan L, Gao M, Liu D. Synthetic FXR agonist GW4064 prevents diet-induced hepatic steatosis and insulin resistance. Pharm Res. 2013 May; 30(5):1447-57. doi: 10.1007/s11095-013-0986-7. Epub 2013 Feb. 1.
Marinozzi M, Carotti A, Sardella R, Buonerba F, Ianni F, Natalini B, Passeri D, Rizzo G, Pellicciari R. Asymmetric synthesis of the four diastereoisomers of a novel non-steroidal farnesoid X receptor (FXR) agonist: Role of the chirality on the biological activity. Bioorg Med Chem. 2013 Jul. 1; 21(13):3780-9. doi: 10.1016/j.bmc.2013.04.038. Epub 2013 Apr. 23.
Misawa T, Hayashi H, Makishima M, Sugiyama Y, Hashimoto Y. E297G mutated bile salt export pump (BSEP) function enhancers derived from GW4064: structural development study and separation from farnesoid X receptor-agonistic activity. Bioorg Med Chem Lett. 2012 Jun. 15; 22(12):3962-6. doi: 10.1016/j.bmcl.2012.04.099. Epub 2012 Apr. 30.
Mudaliar S, Henry R R, Sanyal A J, Morrow L, Marschall H U, Kipnes M, Adorini L, Sciacca C I, Clopton P, Castelloe E, Dillon P, Pruzanski M, Shapiro D. Efficacy and safety of the farnesoid X receptor agonist obeticholic acid in patients with type 2 diabetes and nonalcoholic fatty liver disease. Gastroenterology. 2013 September; 145(3):574-82. el. doi: 10.1053/j.gastro.2013.05.042. Epub 2013 May 30.
Richter H G, Benson G M, Bleicher K H, Blum D, Chaput E, Clemann N, Feng S, Gardes C, Grether U, Hartman P, Kuhn B, Martin R E, Plancher J M, Rudolph M G, Schuler F, Taylor S. Optimization of a novel class of benzimidazole-based farnesoid X receptor (FXR) agonists to improve physicochemical and ADME properties. Bioorg Med Chem Lett. 2011 Feb. 15; 21(4):1134-40. doi: 10.1016/j.bmcl.2010.12.123. Epub 2010 Dec. 31.
Rizzo G, Passeri D, De Franco F, Ciaccioli G, Donadio L, Rizzo G, Orlandi S, Sadeghpour B, Wang X X, Jiang T, Levi M, Pruzanski M, Adorini L. Functional characterization of the semisynthetic bile acid derivative INT-767, a dual farnesoid X receptor and TGR5 agonist. Mol Pharmacol. 2010 October; 78(4):617-30. doi: 10.1124/mo1.110.064501. Epub 2010 Jul. 14.
Schuster D, Markt P, Grienke U, Mihaly-Bison J, Binder M, Noha S M, Rollinger J M, Stuppner H, Bochkov V N, Wolber G. Pharmacophore-based discovery of FXR agonists. Part I: Model development and experimental validation. Bioorg Med Chem. 2011 Dec. 1; 19(23):7168-80. doi: 10.1016/j.bmc.2011.09.056. Epub 2011 Oct. 4.
Soisson S M, Parthasarathy G, Adams A D, Sahoo S, Sitlani A, Sparrow C, Cui J, Becker J W. Identification of a potent synthetic FXR agonist with an unexpected mode of binding and activation. Proc Natl Acad Sci USA. 2008 Apr. 8; 105(14):5337-42. doi: 10.1073/pnas.0710981105. Epub 2008 Apr. 7.
Watanabe M, Horai Y, Houten S M, Morimoto K, Sugizaki T, Arita E, Mataki C, Sato H, Tanigawara Y, Schoonjans K, Itoh H, Auwerx J. Lowering bile acid pool size with a synthetic farnesoid X receptor (FXR) agonist induces obesity and diabetes through reduced energy expenditure. J Biol Chem. 2011 Jul. 29; 286(30):26913-20. doi: 10.1074/jbc.M111.248203. Epub 2011 Jun. 1.
Yu D, Mattern D L, Forman B M. An improved synthesis of 6α-ethylchenodeoxycholic acid (6ECDCA), a potent and selective agonist for the Farnesoid X Receptor (FXR). Steroids. 2012 November; 77(13):1335-8. doi: 10.1016/j.steroids.2012.09.002. Epub 2012 Sep. 21.
Zhang S, Wang J, Liu Q, Hamish D C. Farnesoid X receptor agonist WAY-362450 attenuates liver inflammation and fibrosis in murine model of non-alcoholic steatohepatitis. J Hepatol. 2009 August; 51(2):380-8. doi: 10.1016/j jhep.2009.03.025. Epub 2009 May 18.

Typically FXR agonists include the class of steroid FXR agonists and non steroid FXR agonists.

In some embodiments, the FXR agonist is selected from small molecule compounds which act as FXR modulators that have been disclosed in the following patent publications: EP1392714; EP1568706; EP2128158, EP2289883, JP2005281155; US20030203939; US2005080064; US2006128764; US20070010562; US20070015796; US20080038435; US20080300235; US20090062526, US20090163552, US20100093818, US20100184809; US20110077273, US20110105475; US6984560; US7671085, WO2000037077; WO200040965; WO200076523; WO2001017994; WO2003015771; WO2003016280; WO2003016288; WO2003030612; WO2003060078; WO2003080803; WO2003090745; WO2004007521; WO2004046162; WO2004048349; WO2005082925; WO2005092328; WO2005097097; WO2006020680; WO2007076260; WO2007076260; WO2007092751; WO2007140174; WO2007140183; WO2008000643; WO2008002573; WO2008025539; WO2008025540; WO2008051942; WO2008073825; WO2008157270; WO2009005998; WO2009012125, WO2009027264; WO2009062874, WO2009080555; WO2009127321; WO2009149795, WO2010028981; WO2010034649, WO2010034657, WO2010069604, WO2011020615, WO2013007387, and WO2013037482.

Specific examples of FXR agonists include but are not limited to GW4064 (as disclosed in PCT Publication No. WO 00/37077 or in US2007/0015796), 6-ethyl-chenodeoxycholic acids (6ECDCA), especially 3α, 7α-dihydroxy 7α-dihydroxy-6α-ethyl-513-cholan-24-oic acid, also referred to as INT-747; 6-ethyl-ursodeoxycholic acids, INT-1103, UPF-987, WAY-362450, MFA-1, GW9662, T0901317, fexaramine, a cholic acid, a deoxycholic acid, a glycocholic acid, a glycodeoxycholic acid, a taurocholic acid, a taurodihydrofusidate, a taurodeoxycholic acid, a cholate, a glycocholate, a deoxycholate, a taurocholate, a taurodeoxycho late, a chenodeoxycholic acid, a 7-B-methyl cholic acid, a methyl lithocholic acid.

In some embodiments, the FXR agonist is selected from the group consisting of GW4064, 6ECDCA and the compound identified by the CAS REGISTRY NUMBER 1192171-69-9 (described in WO 2009127321 also named PXL007):

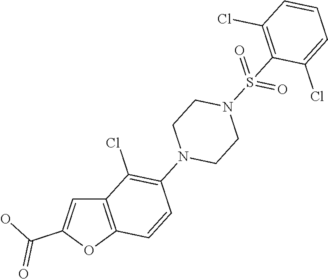 [SMILES: O=C(O)c1cc2c(Cl)c(N3CCN(S(=O)(=O)c4c(Cl)cccc4Cl)CC3)ccc2o1]

In some embodiments, the FXR agonist is the compound having the formula of:

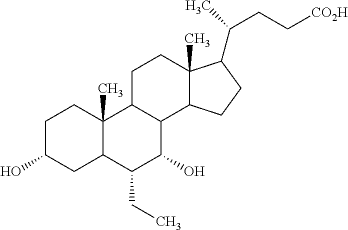 [SMILES: CC[C@@H]1C2C[C@H](O)CC[C@]2(C)C2CC[C@@]3(C)C(CCC3[C@H](C)CCC(=O)O)C2[C@@H]1O]

In some embodiments, the FXR agonist is the compound having the formula of:

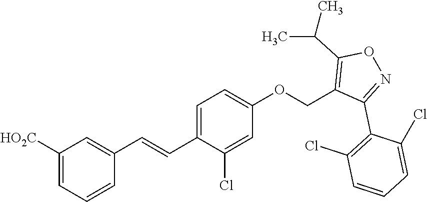 [SMILES: CC(C)c1onc(-c2c(Cl)cccc2Cl)c1COc1ccc(/C=C/c2cccc(C(=O)O)c2)c(Cl)c1]

In some embodiments, the FXR agonist is the compound having the formula of:

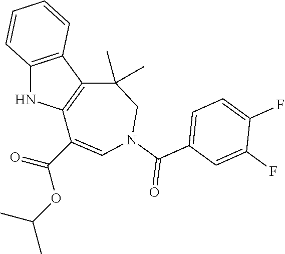 [SMILES: CC(C)OC(=O)C1=CN(C(=O)c2ccc(F)c(F)c2)CC(C)(C)c2c1[nH]c1ccccc21]

In some embodiments, the FXR agonist is obeticholic acid (abbreviated to OCA) which is a semi-synthetic bile acid analogue which has the chemical structure 6α-ethyl-chenodeoxycholic acid. The compound is also known as INT-747.

In some embodiments, the FXR agonist is selected from the group consisting of:

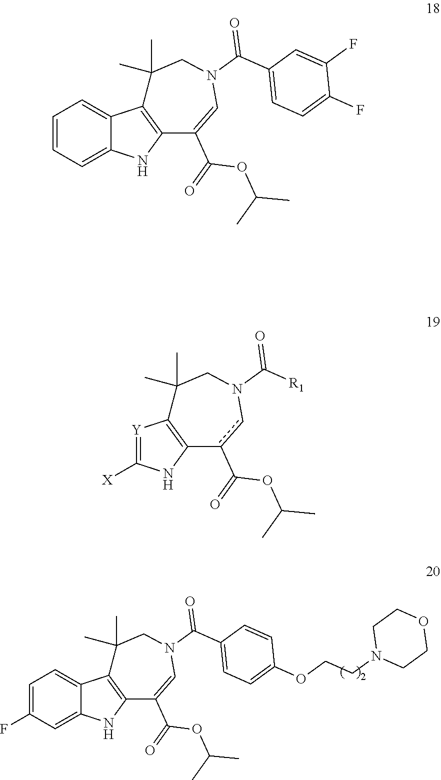

In some embodiments, the FXR agonist is selected from the group consisting of the compounds disclosed in WO2013007387, namely:

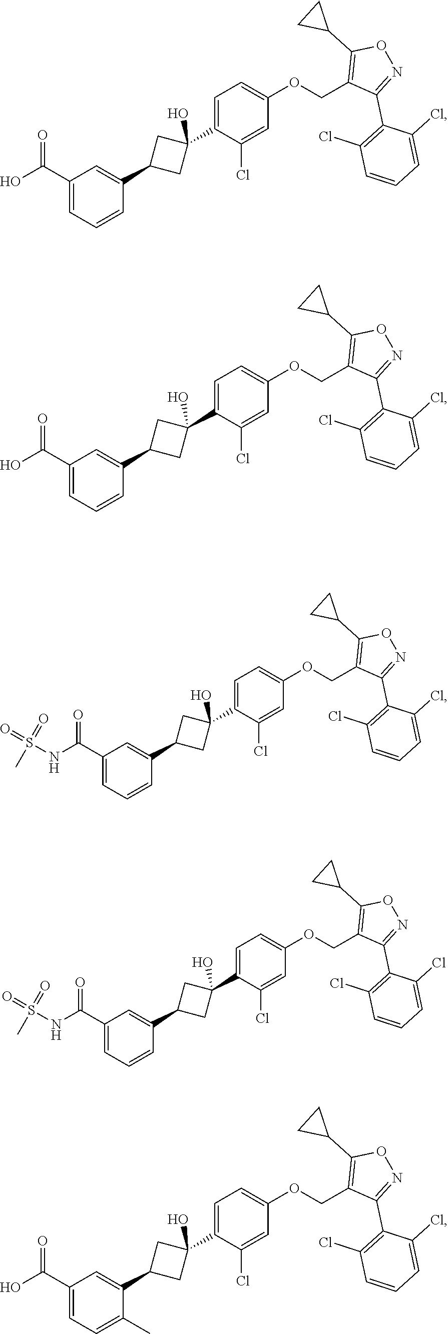 [SMILES: O=C(O)c1cccc([C@H]2C[C@](O)(c3ccc(OCc4c(-c5c(Cl)cccc5Cl)noc4C4CC4)cc3Cl)C2)c1 ; O=C(O)c1cccc([C@H]2C[C@@](O)(c3ccc(OCc4c(-c5c(Cl)cccc5Cl)noc4C4CC4)cc3Cl)C2)c1 ; CS(=O)(=O)NC(=O)c1cccc([C@H]2C[C@](O)(c3ccc(OCc4c(-c5c(Cl)cccc5Cl)noc4C4CC4)cc3Cl)C2)c1 ; CS(=O)(=O)NC(=O)c1cccc([C@H]2C[C@@](O)(c3ccc(OCc4c(-c5c(Cl)cccc5Cl)noc4C4CC4)cc3Cl)C2)c1 ; Cc1ccc(C(=O)O)cc1[C@H]1C[C@](O)(c2ccc(OCc3c(-c4c(Cl)cccc4Cl)noc3C3CC3)cc2Cl)C1]

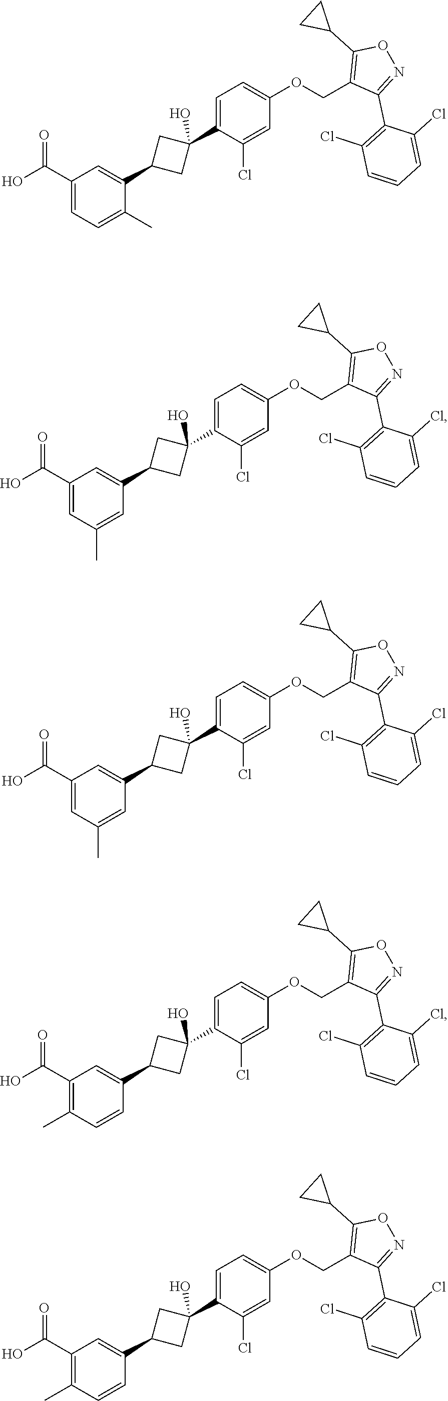 [SMILES: Cc1ccc(C(=O)O)cc1[C@H]1C[C@@](O)(c2ccc(OCc3c(-c4c(Cl)cccc4Cl)noc3C3CC3)cc2Cl)C1 ; Cc1cc(C(=O)O)cc([C@H]2C[C@](O)(c3ccc(OCc4c(-c5c(Cl)cccc5Cl)noc4C4CC4)cc3Cl)C2)c1 ; Cc1cc(C(=O)O)cc([C@H]2C[C@@](O)(c3ccc(OCc4c(-c5c(Cl)cccc5Cl)noc4C4CC4)cc3Cl)C2)c1 ; Cc1ccc([C@H]2C[C@](O)(c3ccc(OCc4c(-c5c(Cl)cccc5Cl)noc4C4CC4)cc3Cl)C2)cc1C(=O)O ; Cc1ccc([C@H]2C[C@@](O)(c3ccc(OCc4c(-c5c(Cl)cccc5Cl)noc4C4CC4)cc3Cl)C2)cc1C(=O)O]

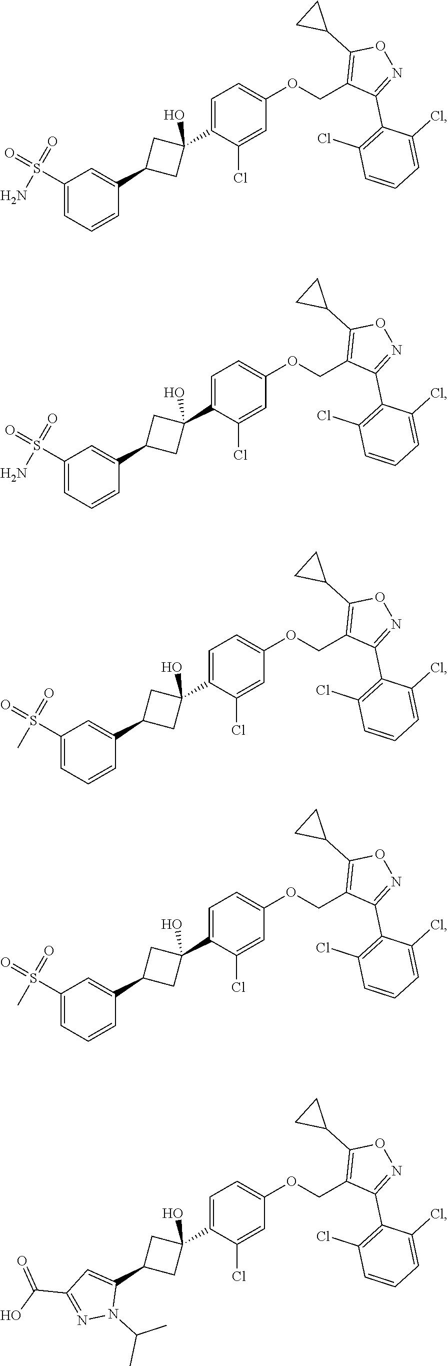 [SMILES: NS(=O)(=O)c1cccc([C@H]2C[C@](O)(c3ccc(OCc4c(-c5c(Cl)cccc5Cl)noc4C4CC4)cc3Cl)C2)c1 ; NS(=O)(=O)c1cccc([C@H]2C[C@@](O)(c3ccc(OCc4c(-c5c(Cl)cccc5Cl)noc4C4CC4)cc3Cl)C2)c1 ; CS(=O)(=O)c1cccc([C@H]2C[C@](O)(c3ccc(OCc4c(-c5c(Cl)cccc5Cl)noc4C4CC4)cc3Cl)C2)c1 ; CS(=O)(=O)c1cccc([C@H]2C[C@@](O)(c3ccc(OCc4c(-c5c(Cl)cccc5Cl)noc4C4CC4)cc3Cl)C2)c1 ; CC(C)n1nc(C(=O)O)cc1[C@H]1C[C@](O)(c2ccc(OCc3c(-c4c(Cl)cccc4Cl)noc3C3CC3)cc2Cl)C1]

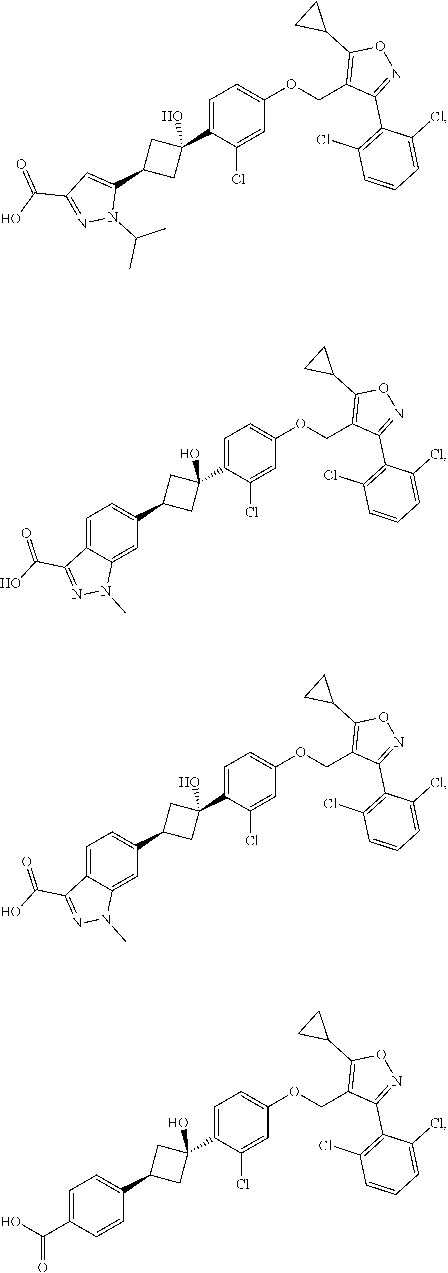 [SMILES: CC(C)n1nc(C(=O)O)cc1[C@H]1C[C@@](O)(c2ccc(OCc3c(-c4c(Cl)cccc4Cl)noc3C3CC3)cc2Cl)C1 ; Cn1nc(C(=O)O)c2ccc([C@H]3C[C@](O)(c4ccc(OCc5c(-c6c(Cl)cccc6Cl)noc5C5CC5)cc4Cl)C3)cc21 ; Cn1nc(C(=O)O)c2ccc([C@H]3C[C@@](O)(c4ccc(OCc5c(-c6c(Cl)cccc6Cl)noc5C5CC5)cc4Cl)C3)cc21 ; O=C(O)c1ccc([C@H]2C[C@](O)(c3ccc(OCc4c(-c5c(Cl)cccc5Cl)noc4C4CC4)cc3Cl)C2)cc1]

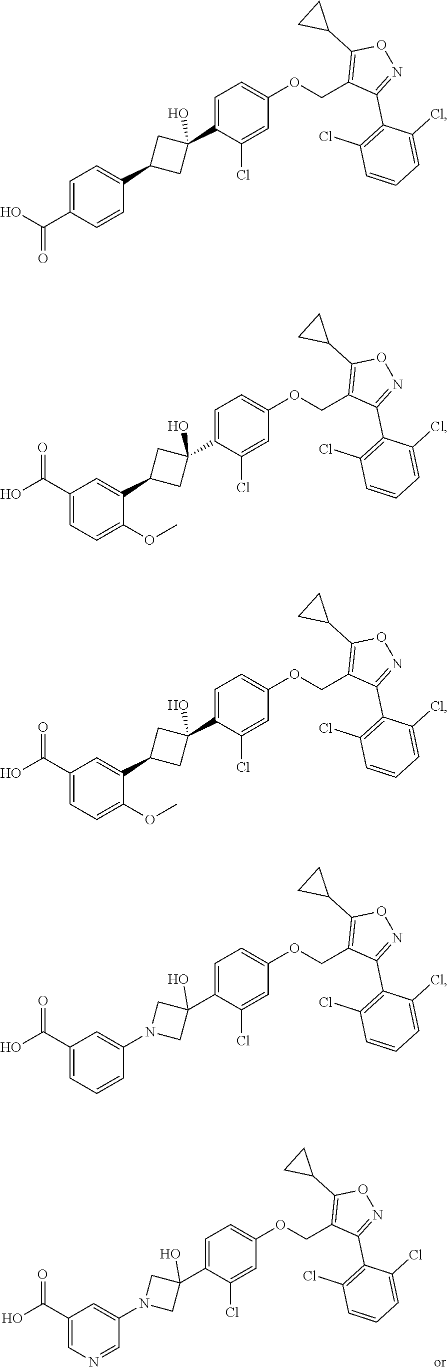 [SMILES: O=C(O)c1ccc([C@H]2C[C@@](O)(c3ccc(OCc4c(-c5c(Cl)cccc5Cl)noc4C4CC4)cc3Cl)C2)cc1 ; COc1ccc(C(=O)O)cc1[C@H]1C[C@](O)(c2ccc(OCc3c(-c4c(Cl)cccc4Cl)noc3C3CC3)cc2Cl)C1 ; COc1ccc(C(=O)O)cc1[C@H]1C[C@@](O)(c2ccc(OCc3c(-c4c(Cl)cccc4Cl)noc3C3CC3)cc2Cl)C1 ; O=C(O)c1cccc(N2CC(O)(c3ccc(OCc4c(-c5c(Cl)cccc5Cl)noc4C4CC4)cc3Cl)C2)c1 ; O=C(O)c1cncc(N2CC(O)(c3ccc(OCc4c(-c5c(Cl)cccc5Cl)noc4C4CC4)cc3Cl)C2)c1 ; "or"]

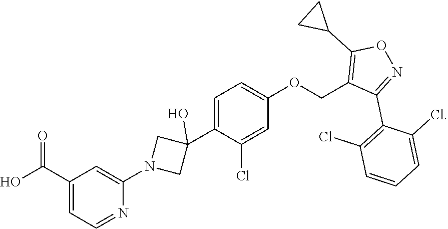 [SMILES: O=C(O)c1ccnc(N2CC(O)(c3ccc(OCc4c(-c5c(Cl)cccc5Cl)noc4C4CC4)cc3Cl)C2)c1]

In some embodiments, the FXR agonist is selected from the group consisting of the compounds disclosed in WO2009149795, namely:

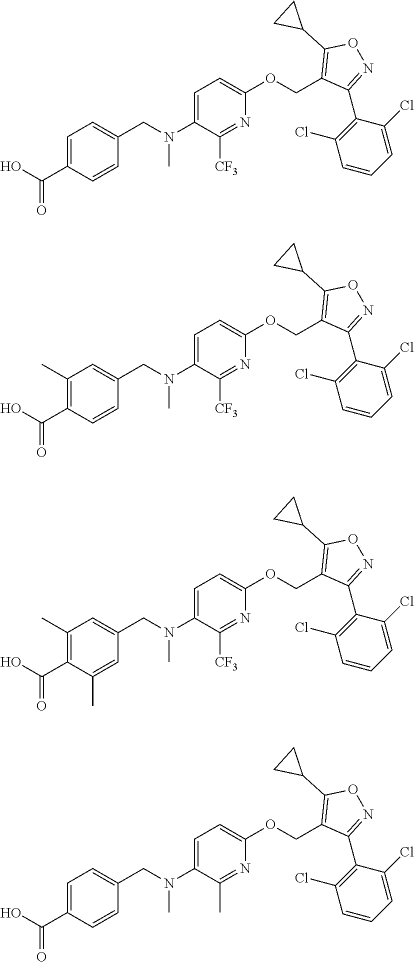 [SMILES: CN(Cc1ccc(C(=O)O)cc1)c1ccc(OCc2c(-c3c(Cl)cccc3Cl)noc2C2CC2)nc1C(F)(F)F ; Cc1cc(CN(C)c2ccc(OCc3c(-c4c(Cl)cccc4Cl)noc3C3CC3)nc2C(F)(F)F)ccc1C(=O)O ; Cc1cc(CN(C)c2ccc(OCc3c(-c4c(Cl)cccc4Cl)noc3C3CC3)nc2C(F)(F)F)cc(C)c1C(=O)O ; Cc1nc(OCc2c(-c3c(Cl)cccc3Cl)noc2C2CC2)ccc1N(C)Cc1ccc(C(=O)O)cc1]

In some embodiments, the FXR agonist is selected from the group consisting of the compound disclosed in WO2008025539, namely:

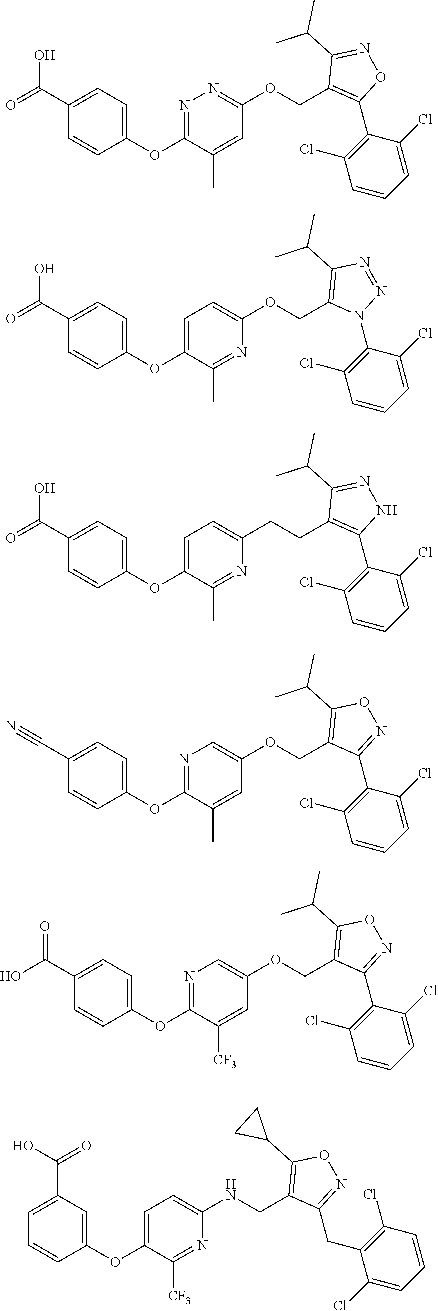

In some embodiments, the FXR agonist is selected from the group consisting of the compounds described in WO2008025540, namely:

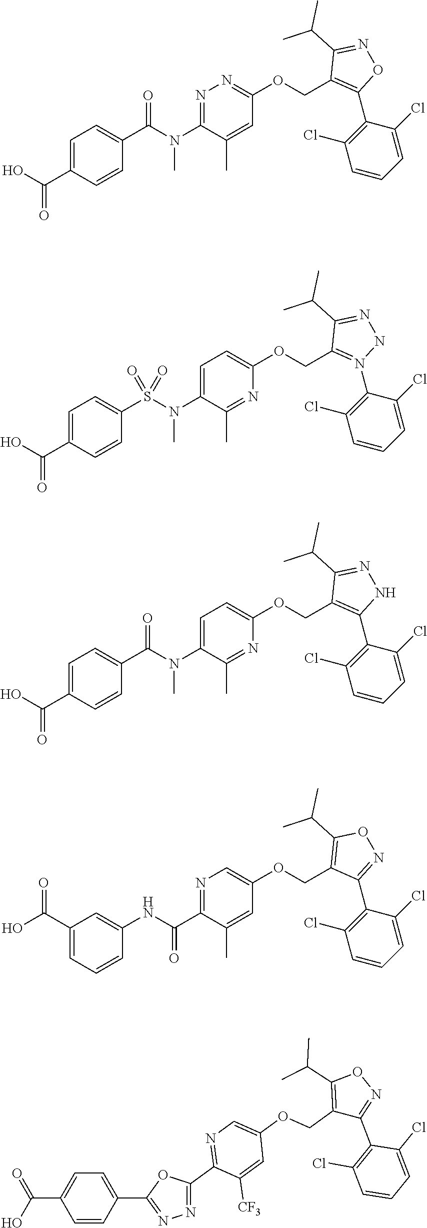

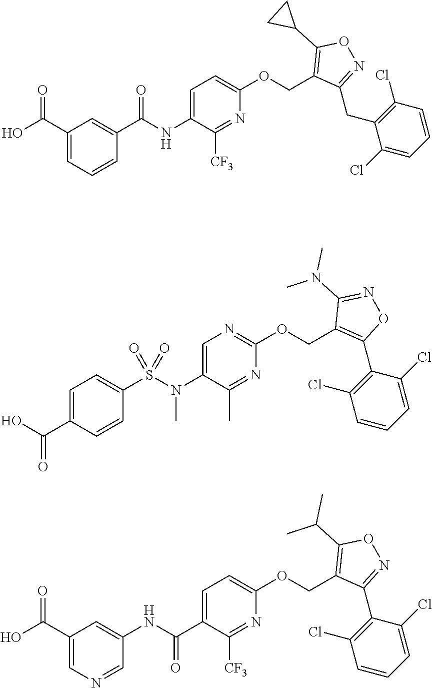 [SMILES: O=C(O)c1cccc(C(=O)Nc2ccc(OCc3c(Cc4c(Cl)cccc4Cl)noc3C3CC3)nc2C(F)(F)F)c1 ; Cc1nc(OCc2c(N(C)C)noc2-c2c(Cl)cccc2Cl)ncc1N(C)S(=O)(=O)c1ccc(C(=O)O)cc1 ; CC(C)c1onc(-c2c(Cl)cccc2Cl)c1COc1ccc(C(=O)Nc2cncc(C(=O)O)c2)c(C(F)(F)F)n1]

Additional FXR agonists useful in the present inventions can be identified routinely by those of skill in the art based upon assays such as described in PCT/US99/30947, the teachings of which are herein incorporated by reference in their entirety. Typically, FXR agonists are identified using a nuclear receptor-peptide assay. This assay utilizes fluorescence resonance energy transfer (FRET) and can be used to test whether putative ligands bind to FXR. The FRET assay is based upon the principle that ligands induce conformational changes in nuclear receptors that facilitate interactions with coactivator proteins required for transcriptional activation. In FRET, a fluorescent donor molecule transfers energy via a non-radioactive dipole-dipole interaction to an acceptor molecule (which is usually a fluorescent molecule. Alternatively, activity of FXR ligand is identify by monitoring the effect of these ligands on expression of a reporter gene under the control of a promoter that contains one or several copies of typical consensus FXR responses elements. This later assay relies on plasmid construct that contains a promoter in front of a reporter gene, typically a luciferase gene, and that can be easily amplified in bacteria and transfected in mammalian cells. This assay can globally assess the consequences of ligands on FXR transcriptional activity, which can be positive (agonist) or negative (antagonist).

Typically the FXR agonist of the invention is administered to the subject with a therapeutically effective amount. By a “therapeutically effective amount” of the FXR agonist as above described is meant a sufficient amount of the FXR agonist to treat the viral infection at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination with the specific agonist employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The FXR agonist of the invention may be combined with pharmaceutically acceptable excipients, and optionally sustained-release matrices, such as biodegradable polymers, to form therapeutic compositions. “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Galenic adaptations may be done for specific delivery in the small intestine or colon. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising FXR agonists of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The FXR agonist of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifusoluble agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. The FXR agonist of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. In addition to the FXR agonists of the invention formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### FIGURES

FIG. 1: Effect of FXR ligands on H9 cell line proliferation and cell death. H9 cell were grown in standard RPMI1640 starting at 1×10⁶ cells/mL and treated or not with GW4064 or Takeda at 10 μM, or DMSO vehicle only. After cell count and viability were determined three times a week by Cellometer Nexcelom Auto 1000 device, a volume of cell culture (cells and medium) was removed and replaced by fresh medium with molecules to maintain the cell concentrations around 1×10⁶ cells/mL. Cell proliferation was defined by the cell proliferation factor that calculated the number of cells derived from one seeding cell at each medium change (upper panel). Dosage of LDH activity released in the medium monitored cell death rate (lower panel).

FIG. 2: Effect of FXR ligands on activated PBMC survival and death. PHA/IL2 activated PBMC were seeded at 1×10⁶ cells/mL in standard RPMI1640 supplemented with IL2 and treated or not with GW4064 or Takeda at 10 μM, or DMSO vehicle alone and keep for one week. Aliquots were taken and cell counted using Cellometer Nexcelom Auto 1000 device and cell proliferation factor was calculated as in FIG. 1 at each medium change (upper panel). Dosage of LDH activity released in the medium monitored cell death rate (lower panel).

FIG. 3: Effects of FXR ligands on cell viability and p24 production in HIV-1 infected H9 cells. 30 millions H9 cells were incubated in 10 mL of 1/100 dilution of HIV-1 NL4.3 virus stock in standard RPMI for 6 hours. Cells were then washed twice in RPMI and seeded at 1×10⁶ cells per mL in standard RPMI and treated with GW4064 or Takeda, both at 10 μM or DMSO vehicle only in 10 mL cultures. At the indicated time, cell count and viability were determined by Cellometer Nexcelom Auto 1000 device and a volume of cell culture (cells and medium) was removed and replaced by fresh medium with molecules to maintain the cell concentration around 1×10⁶ cells/mL. Cell proliferation as defined by the number of cells from one seeding cell was calculated at each medium change. Removed cell free medium was stored at −20° C. until infectious titer determination and p24 dosage. Upper and middle panels show cell proliferation and percentage of cell viability respectively in each condition at the indicated time post-infection and lower panel, the cumulative production of p24.

FIG. 4: Effects of FXR ligands on HIV-1 virus production by HIV-1 infected H9 cells. Cells were prepared and treated as in FIG. 3. Upper and middle panels show the infectious titers, the actual number of infectious particles per mL of cell free culture medium, for each condition at the indicated time post-infection, plotted with a logarithmic or linear Y scale respectively. Lower panel shows the ratio of infectious particles per ng of p24.

FIG. 5: FXR agonist GW4064 dose-responses on HIV-1 replication in infected H9 cells. Cells were treated and infected as in FIG. 3 and the effects of GW4064 at 0.2, 1 and 5 μM, and DMSO vehicle only were tested on cell viability (A), production of infectious virions, infectious titer (B) and specific infectivity defined by the ratio of infectious units per ng of p24 (C) at the indicated time.

FIG. 6: Effects of FXR ligands on HIV-1 replication in activated PBMC from one donor. 30 millions of PHA and IL2 activated PBMC were incubated in 10 mL of 1/100 dilution of HIV-1 NL4.3 virus stock in standard RPMI for 6 hours. Cells were then washed twice in RPMI and seeded at 1×10⁶ cells per mL in standard RPMI supplemented with IL2 and treated with GW4064 or Takeda, both at 10 μM, or DMSO vehicle only. Aliquots were sampled at the indicated time post-infection and stored at −20° C. until infectious titer determination and p24 dosage. Upper panel shows total cell count evolution with time that remained stable for all conditions. Middle panel indicates the cumulative p24 production at the indicated times and lower panel the infectious titer as infectious particles per mL.

FIG. 7: Effects of FXR ligands on HIV-1 replication in activated PBMC from a second donor. Activated PBMC were processed as in FIG. 6 except that fresh RPMI supplemented with IL2 and the indicated molecules was added at day 7. Upper panel shows variations in the total cell counts with time and indicated FXR ligands or vehicle. Middle panel illustrates the evolution of cell viability in the three experimental conditions and lower panel plots the p24 production with a logarithmic Y scale.

FIG. 8: H9 and PBMC express FXR. Whole lysates of H9 and fresh or PHA-IL2 activated PBMC were analyzed by Western blot for presence of FXR. Same amounts of cell lysates were deposited on gel and actin staining showed similar intensity of the actin band. A band corresponding to FXR was detected in H9 and activated PBMC lysates but only faintly in fresh PBMC. FXR expression seems higher in H9 than in PBMC.

FIG. 9: Generation of a FXR-silenced H9 cell line with a shFXR lentiviral vector. Cells were transduced with the shFXR lentiviral vector and selected as described in materials and methods section. Transduced and control cells were cultured for 3 days in presence or absence of GW4064 at 10 μM. Cells were then lysed and analyzed by western blotting for FXR expression. FXR expression is significantly decreased and is further inhibited by the FXR agonist GW4064.

FIG. 10: FXR silencing in H9 cell suppresses the effect of FXR agonist on HIV-1 NL4.3 replication. shFXR and shCont H9 cell were infected with NL4.3 stock virus at 1/100 dilution for 6 hrs. Cells were then washed and seeded at 1×106 cells/mL with fresh medium supplemented with vehicle only or GW4064 at 1 μM and 5 μM (for infectious titers determination). At the indicated days, cells were counted and a calculated volume of cell suspension was removed from each condition to keep cells concentration around 1×10⁶/mL. An equal volume of fresh medium with molecules was added in cell vials. p24 concentrations (A), and HIV titers (B) were determined at each time point for every conditions. p24 and HIV titers are expressed relatively to the values assessed in the DMSO treated shCont H9 at 4, 7 and 9 days. Numbers of asterisks above bars reflect statistical p values; **p<0.01, ***p<0.001 and ****p<0.0001

FIG. 11: Effect of FXR agonist GW4064 on latently infected 8.4 and 15.4 J-Lat clones. The 8.4 and 15.4 clones that contain one silent integrated proviral copy per cell were stimulated with TNFα alone at 0.5 ng/mL or in combination of the FXR agonists GW4064 or 6ECDCA at 5 μM. Proportions of cells that expressed GFP as a consequence of provirus reactivation was quantified by flow-cytometry (A1 and A2). Cell viability and p24 production are shown in panel B1 and B2, and C1 and C2 respectively.

FIG. 12: The FXR agonist GW4064 represses BKV replication in RPTEC. RPTEC cells were seeded in 24 well plates at 5×10⁴ cells/well and maintained in REBM plus 2% FCS but without supplements. Cells were infected with BKV stock dilution (1/100) for 4 hours, and then washed in DMEM. 1 mL/well of REBM plus 2% FCS and GW4064 or vehicle was added. BKV production in the cell supernatants was monitored by qPCR at day 3 and 5 post-infection. Treatment with GW4064 at 10 μM reduced by more than 1.5 Log 10 the virus titer in supernatants compared to mock treated cells at both days post-infection (p<1×10⁻⁴). Standard deviations were less than 2% and are then within the marks.

### EXAMPLE

Material & Methods

Cell Lines

The H9 cell line is a clonal derivative of the T lymphoma Hut 78 cell line selected for permissiveness for HIV-1 replication. Cells were grown at 37° C. under 5% CO2 at concentrations ranging from 0.5 to 1.5×10⁶ cells/mL in RPMI-1640 medium supplemented with 10% fetal calf serum, non essential amino-acids and antibiotics (standard RPMI).

HEK293T is a derivative of the human embryonic kidney 293 cell line into which the temperature sensitive gene for SV40 T-antigen was inserted. HEK293T cell line was maintained in Dulbecco's modified Eagle's medium (DMEM) supplemented with 10% fetal calf serum at 37° C. under 5% CO2.

The Vero cell line was initiated from the kidney of a normal adult African green monkey. Vero cells were maintained in Dulbecco's modified Eagle's medium (DMEM) supplemented with 10% fetal calf serum at 37° C. under 5% CO2.

HeLa-P4 cells expressing human CD4 and CXCR4, HeLa CD4+HIV-1 LTR-β-gal Cells (MAGI), were maintained in DMEM supplemented with 10% fetal calf serum at 37° C. under 5% CO2 (1).

TZM-bl, previously designated JC53-bl (clone 13) is a HeLa cell line. The parental cell line (JC.53) stably expresses large amounts of CD4 and CCRS. The TZM-bl cell line was generated from JC.53 cells by introducing separate integrated copies of the luciferase and β-galactosidase genes under control of the HIV-1 promoter. The TZM-bl cell line is highly sensitive to infection with diverse isolates of HIV-1. Cells were maintained in DMEM supplemented with 10% fetal calf serum at 37° C. under 5% CO2.

Latently HIV-infected clones 8.4 and 15.4 are derived from latent population of jurkat transfected with full-length HIV genome expressing green fluorescent protein that do not express HIV but which can be transcriptionally activated by phorbol esthers or TNFα (2).

Primary human renal proximal tubule epithelial cells (RPTEC) were acquired from Lonza (Swiss). RPTEC are isolated from human kidney proximal tubule and are a mixture of epithelial cells from the cortex and glomerular. Cells were grown following provider's protocols in renal epithelial basal medium (REBM) with supplements and growth factors (hydrocortisone, hEGF, FBS, epinephrine, insulin, triiodothyronine, transferrin and gentamicin/amphotericin-B) from Lonza (Swiss).

Cell viability and counts were measured by trypan blue dye exclusion with Cellometer Nexcelom Auto 1000 (Bioscience) following the manufacturer's recommendations.

Cell death was monitored by measuring the activity of intracellular lactate deshydrogenase (LDH) released in the cell culture medium with the Cytotoxicity Detection kit PLUS (Roche).

Preparation of Human peripheral blood mononuclear cells.

Human peripheral blood mononuclear cells (PBMC) were prepared from blood donations from healthy donors collected at the “Etablissement Francais du Sang”, Lyon. Briefly, an equal volume of PBS with 1 mM EDTA was added to blood. 35 mL diluted blood was then layered on 15 mL of Ficoll (Eurobio) in 50 mL tubes and centrifuged 20 min at 20° C. and 625 g. Cell rings of PBMC at the interface were collected, washed 3 times in PBS. PBMC were then run on a Percoll gradient (GE Healthcare); 3 mL PBMC (25 to 30×10⁶ cells/mL) were layered on 6 mL Percoll 50% in 15 mL tube. After centrifugation for 20 min at 20° C. and 770 g, pellets mainly constituted of T and B lymphocytes and NK cells were collected, washed twice in PBS, resuspended and adjusted to 1×10⁶ cells/mL in RPMI 1640 supplemented with 10% fetal calf serum, non essential amino-acids (NEAA) and antibiotics (standard medium).

Lymphocytes Activation

PBMC, 1×10⁶ cells/mL, were activated in standard medium supplemented with PHA 10 μg/mL and IL2 20 U/mL for 48 h. Medium was then replaced by standard medium with 20 U/mL IL2 at 1×10⁶ cells/mL. Stimulated lymphocytes were then maintained in standard medium with 20 U/mL IL2 during infection and treatment.

Lentiviral Vectors, shRNA and Generation of shFXR and shContFXR H9 Cell Lines

HEK293T cells were plated on 10 cm culture dishes coated with 0.01% L-polylysine (P4832 Sigma) and transfected with 2.5 M CaCl2 at 50% confluency with a plasmid mix of 8 μg pPAX2, 4 μg pVSVG, and 10 μg pLKO.1-puro-shFXR or 10 μg control pLKO.1-puro-shMafG (Table 1). Cells were washed 6 hours later and medium was replaced with reduced serum medium (Opti-MEM, Thermo Fisher Scientific) for 40 hours. Lentiviral particles were concentrated from the clarified supernatant (0.45 μm filter) by centrifugation for 20 minutes at 4500 rpm in Vivaspin 20 (VS2042 Sartorius).

H9 cells were transduced three days after being plated with concentrated shControl or shFXR lentiviral particles for 24 hours. Then cells were washed and medium was replaced with complete RPMI medium for further 24 hours. On the next day, medium was replaced with standard medium containing 3 μg/ml puromycin. The 3 μg/ml puromycin selection was maintained till the end of experiment.

Viruses

Plasmid pNL4-3 is an infectious molecular clone of the T-cell-tropic isolate NL4-3 (3). Virus stocks were prepared in HEK 293T cells transfected with plasmid DNAs using JetPEI Polyplus-Transfection (Ozyme) reagent following the manufacturer's procedure. Virus-containing supernatants at day 3 post-transfection were clarified by centrifugation (1,000×g, 5 min) and filtered through a 0.45-μm-pore-size filter to remove residual cells and debris. Virus stock was aliquoted, stored at −20° C. and titrated after thawing on MAGI cells with standard procedure.

Polyomavirus BKV strain Dunlop was amplified in Vero cell as described in (4). Stock virus titration was performed by qPCR, BK Virus R-gene®, Argene (bioMérieux), and measured at 1×10¹¹ Genomes equivalent/mL.

Chemicals

GW4064 [3-(2,6-dichlorophenyl)-4-(3-carboxy-2-chloro-stilben-4-yl)-oxymethyl-5-isopropyl isoxazole] is a FXR agonist (EC50 90 nM), active both in vivo and in vitro (5). Although displaying a limited bioavailability, GW4064 has gained a widespread use as a powerful and selective FXR ligand and has reached the status of “reference compound” in this field. 6-ECDCA (6-ethyl-cheno-deoxycholic acide) is a bile salt derivative and strong FXR agonist (EC50 99 nM) and was obtained from Sigma-Aldrich (6). Synthetic FXR antagonist CAS936123-05-6, herein referred to as Takeda (described in patent WO 2007052843 A1 20070510; Takeda Pharmaceuticals, Osaka, Japan), was synthesized by Edelris, Lyon, France. These compounds were dissolved in DMSO at 10 mM. Lectin from phasolus vulgaris (PHA-M), human recombinant interleukin 2 (IL-2), and TNFα were purchased from Sigma-Aldrich.

Western Blot

Cells (H9 and PBMC) were washed with PBS and pelleted. Cell pellets were dissolved in lysis buffer (Tris-HCl (pH 7.4), EDTA 1 mM, NaCl 180 mM, 0.5% NP-40 and protease inhibitors) at 4° C. for 20 minutes. The suspension were then centrifuged at 17 000 g for 20 minutes at 4° C. to prepare whole cell extracts. Protein concentrations were determined by the Bradford assay.

Cell lysates were deposited on a 4-12% Bis-Tris Mini Gel (NuPAGE®) into a 1×MES buffer (NuPAGE® MES SDS Running Buffer). After electrophoresis at 200V for 35 minutes, proteins were transferred onto nitrocellulose membranes following the iBlot® Gel Transfer Stacks Nitrocellulose (Invitrogen) protocol. The membranes were saturated for 2 hours in PBS Tween 20 0.1%-0% fat Milk 5%. Then, the membranes were probed with anti-human FXR/NR1H4 monoclonal antibody at 1 μg/ml (R&D Systems) for 1 hour at room temperature, reacted with peroxidase-conjugated AffiniPure Goat Anti-Mouse IgG (Jackson ImmunoResearch Laboratories) at 0.08 μg/ml for 1 hour at room temperature using Super Signal® West—Maximum Sensitivity Substrate (ThermoFisher Scientific).

HIV-1 p24 Quantification

p24 quantification was performed with the HIV P24 II kit using a mini VIDAS automated device (bioMérieux) following the manufacturer's instructions. Culture medium was centrifuged and cell free supernatants were inactivated before dosage by addition of an equal volume of PBS with 4% Tween 20.

HIV-1 Titration

Titration of infectious units produced by infected cells was performed using the MAGI cells or TZM-bl cells. Briefly, serially diluted supernatants were distributed in 24-well plate containing 1 to 4×10⁵ MAGI cells per well or in 96-well plate containing 1 to 4×10⁴ TZM-bl cells per well. After 2 days of culture, cells were fixed and stained for beta-galactosidase expression and blue-stained positive cells were counted by microscopic examination. Infectious titers were then expressed as infectious units per mL of supernatant. Alternatively, with the TZM-bl cells after 2 days of culture cell were lysed and chemiluminescence activity was read using an illuminometer. Control cell supernatant were treated in parallel and baseline luminescence was subtracted from recorded values from infected supernatants. Infectious titers were then expressed as luminescence activity in arbitrary units.

Results

Treatment with FXR modulators does not alter lymphoblastic H9 or PBMC cell survival and proliferation.

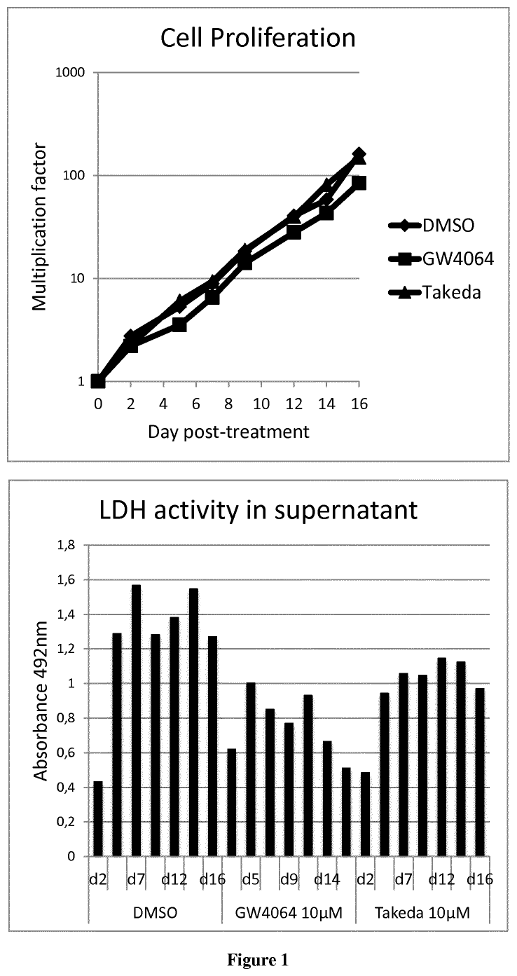

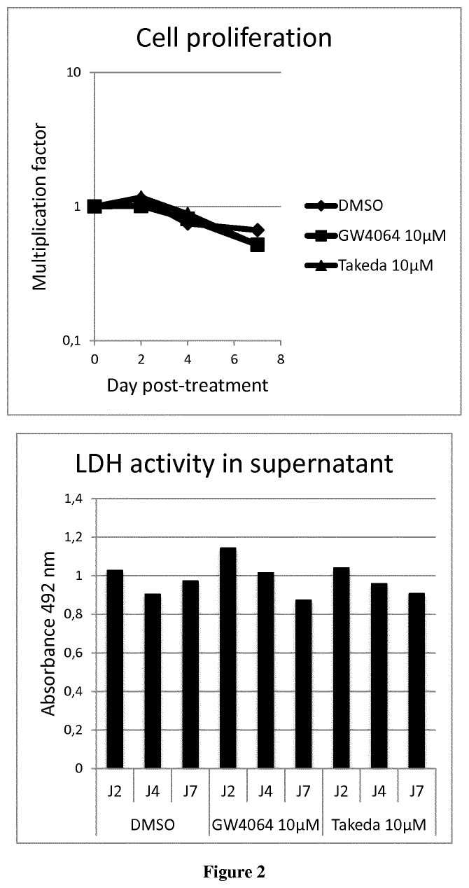

The effect of FXR ligands on proliferation or survival of the lymphoblastoid cell line H9 and of PHA-IL2 activated PBMC was first tested. Proliferation of H9 was not altered by exposition to FXR ligands, the agonist GW4064 and antagonist Takeda, at 10 μM for two weeks (FIG. 1, upper panel) nor it was observed significant modification of the release of lactate deshydrogenase (LDH) (FIG. 1 lower panel) in the cell culture medium. Surprinsingly, LDH release into cell supernatant seemed even lower in presence of GW4064, suggesting a protective effect of GW4064 for H9 cells. Similarly, FXR ligands did not modify the number of activated PBMC that remained stable over the week of observation nor induce cell lysis (FIG. 2). Therefore, treatment with both FXR ligands did not significantly modify the H9 and PBMC proliferation-survival or cell death rate.

Treatments with FXR ligands modulate HIV-1 replication and cell survival in lymphoblastic cell line H9 and PBMC.

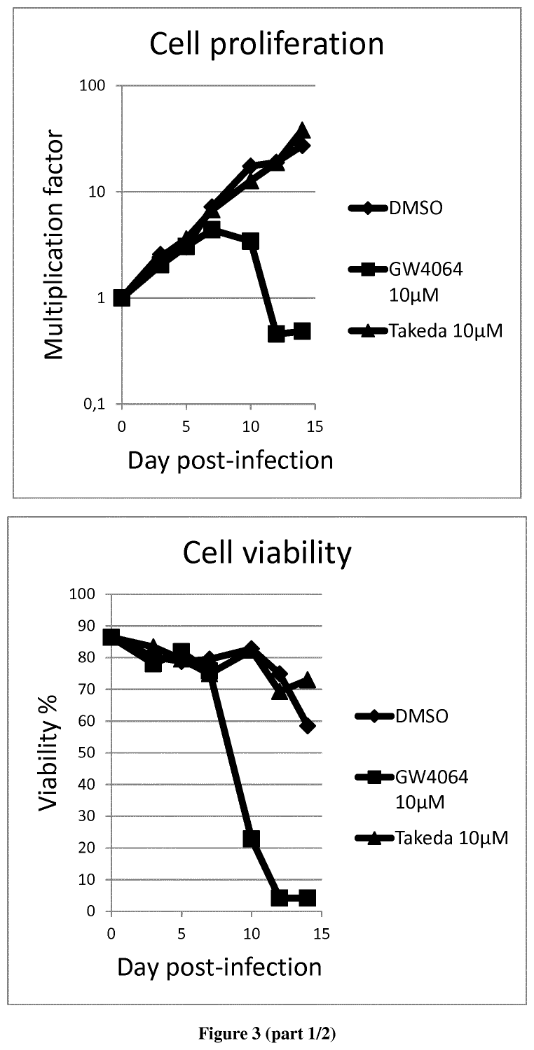

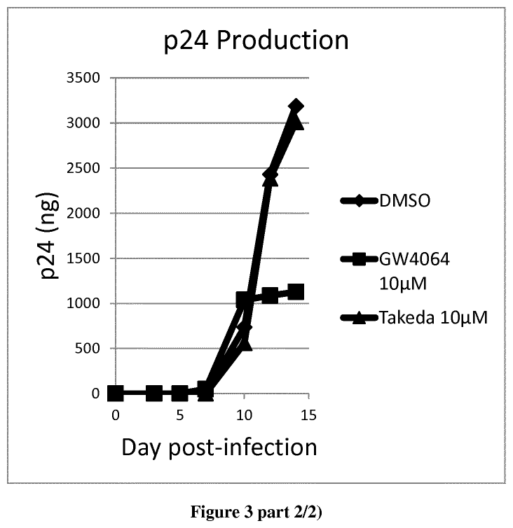

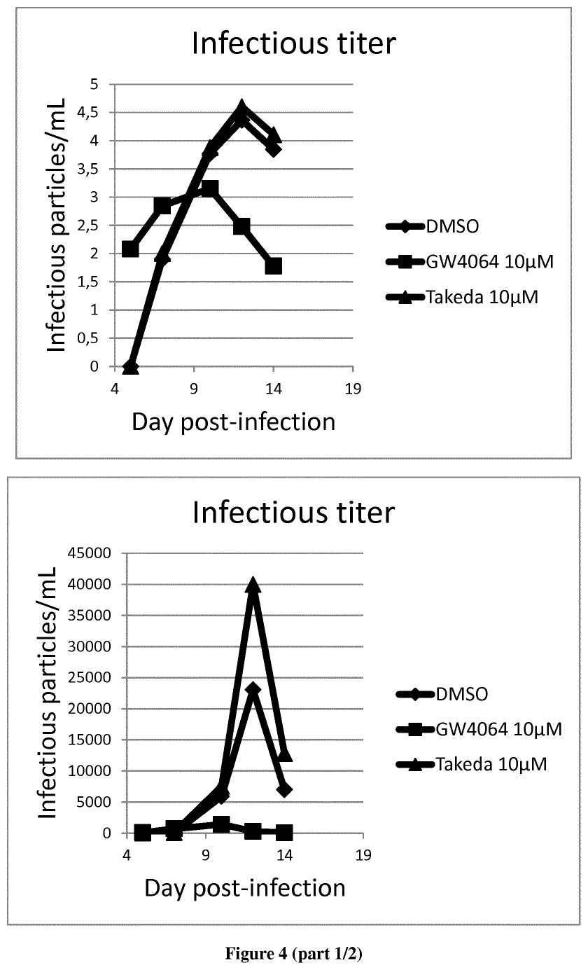

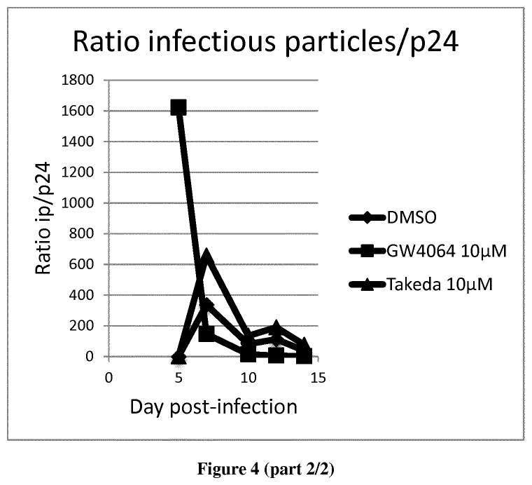

The effects of treatment with FXR agonist and antagonist on HIV-1 NL4.3 infected lymphoblastic cell line H9 were then tested. Agonist GW4064 had a strong impact on H9 proliferation and viability (FIG. 3 upper and middle panel); indeed cell number and percentage of viable cells decreased dramatically compared to mock treated cells or cells treated with the antagonist. Concomitantly, total production of HIV-1 p24 by GW4064 treated cells reached a plateau at day 7 post-infection while it continued to increase in the two other conditions (FIG. 3 right panel). Finally, the kinetics of infectious titers as measured by the number of infectious particles/mL of medium varied greatly depending on the treatment. Agonist GW4064 induced a quick burst of infectious particles that was detected as soon as day 5 with a following decline after the initial peak (FIG. 4 upper and middle panel). Kinetics of mock or Takeda treated cells showed a slower increase in the production of infectious particles that peaked later, at day 12 post-infection, and at a higher level than agonist treated cells. Interestingly (FIG. 4 lower panel) the number of infectious particles per p24 ng showed that GW4064 treated cell produced infectious particles very early and efficiently when production of infectious viral particles peaked later and at a lower level by cells in the two other conditions. Altogether, these data indicate that FXR agonist induces a brief and efficient boost of viral replication that is quickly followed by massive cell death. These effects overall considerably reduce the viral replication compared to cells treated with FXR antagonist or vehicle.

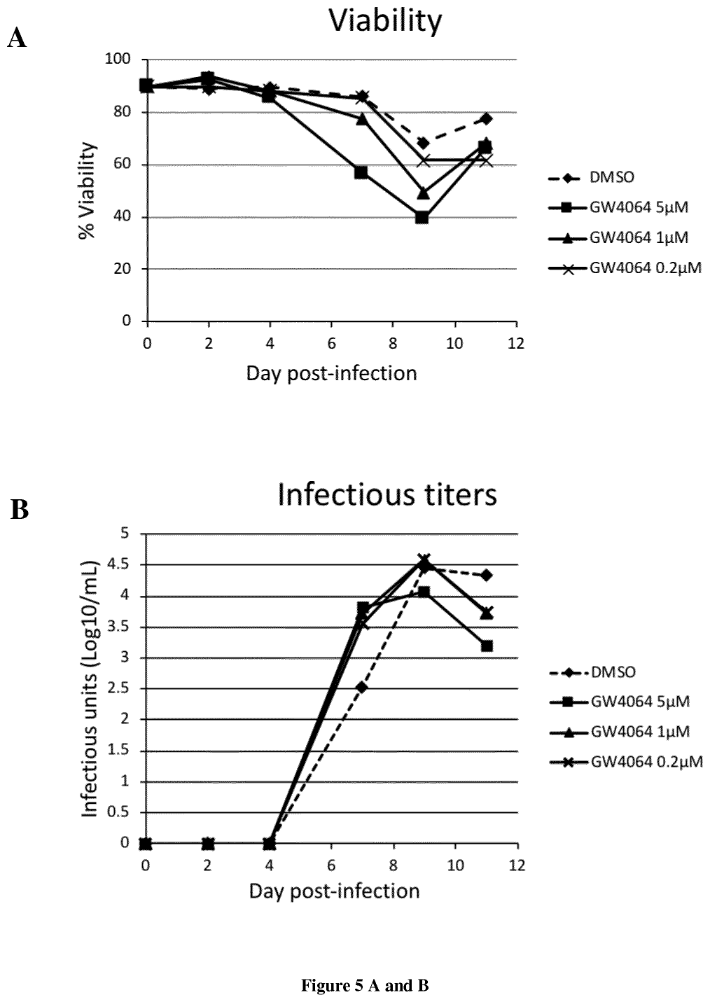

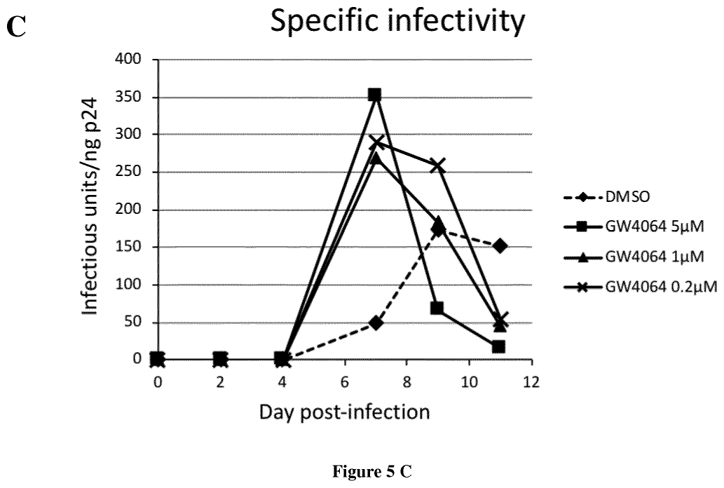

The effects of FXR agonist on HIV NL4.3 replication are dose-dependent. Cell viability decreased in a dose-dependent manner from almost 70% with vehicle only to 40% when treated with 5 μM GW4064 (FIG. 5A). Concentration as low as 0.2 μM was sufficient to induce an effect on cell viability. Rebounds of viability in all conditions at day 11 likely reflect on-going cell proliferation of uninfected cells or newly infected cells before further cycles of viral replication can impact cell survival. Increasing concentrations of GW4064 boosted production of infectious particles by more than one Log 10 at all GW4064 dosings compared to mock treated cells at day 7 (FIG. 5B). Production of infectious particles then dropped below the production by mock treated cells in a dose-dependent manner and reached minus one Log 10 at 5 μM. Finally, increasing GW4064 concentrations had a dose-dependent biphasic effect on the specific infectivity as described by the ratio of infectious particles per ng of p24 (FIG. 5C). There was first an early and dose-dependent increase of specific infectivity compared to mock treated cells, and latter a sharp decline at day 9 and 11. Interestingly, the kinetic of the decline seemed slower as doses decreased. However, overall GW4064 repressed HIV-1 replication in a dose-dependent manner.

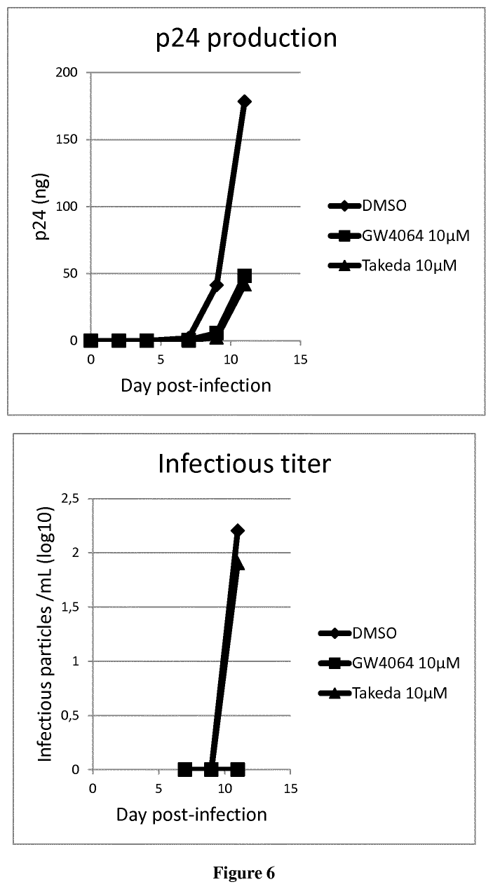

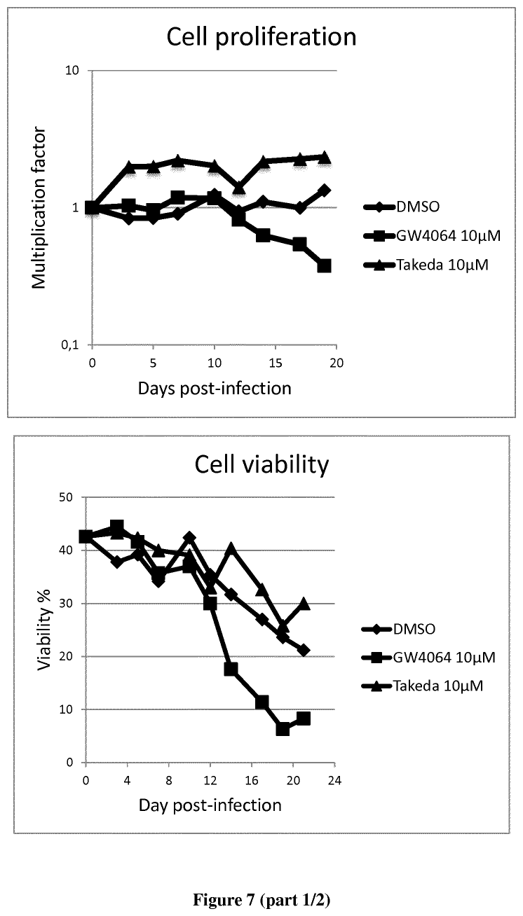

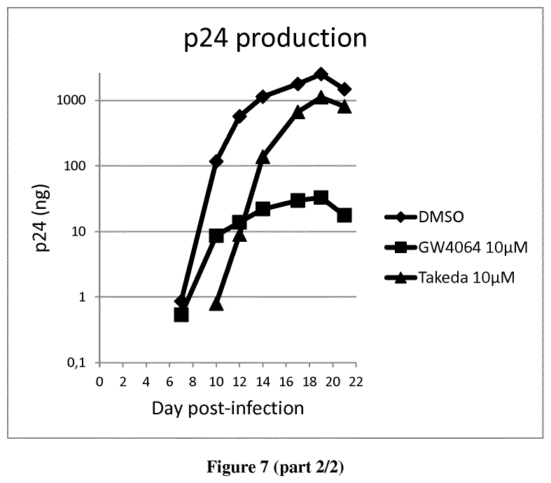

Similar experiments were performed with PHA-IL2 activated PBMC from healthy donors. Data obtained with PBMC prepared from two different donors are shown here as examples. With PBMC from a first donor, HIV-1 replication could not be detected before day 7 post-infection and increased more significantly in GW4064 treated cells than in the two other conditions at day 9 (FIG. 6). However, if the highest p24 production was observed in GW4064 treated cell culture, no infectious virions could be observed in the conditions of the titration assay. Indeed, production of infectious viral particles was only detected in cells incubated with Takeda or vehicle. With PBMC from a second donor (FIG. 7), infection was detected earlier, as soon as day 7, and p24 production reached higher levels than with the first donor at the same days post-infection. Of note, treatment with Takeda delayed p24 production in comparison with the other conditions. Interestingly, number of PBMC during the observation period decreased with time only in presence of GW4064 and the decrease of the percentage of viable cells was more important also when cultures were treated with the FXR agonist. These data indicated that treatment with GW4064 is associated with a higher rate of cell death in HIV-1 infected PBMC. Again, infectious particles were detected only in mock or Takeda treated cultures and not in GW4064 ones (not shown) in the condition of the assay. The difference of the replication kinetics between the two donors likely reflects variation of the donor cells permissiveness to HIV-1 infection. Altogether, these data confirmed those obtained with the H9 cell line, with an early and quick boost of p24 production that is then followed by cell death and, overall, a strong reduction of production of infectious particles.

H9 and PBMC Express FXR

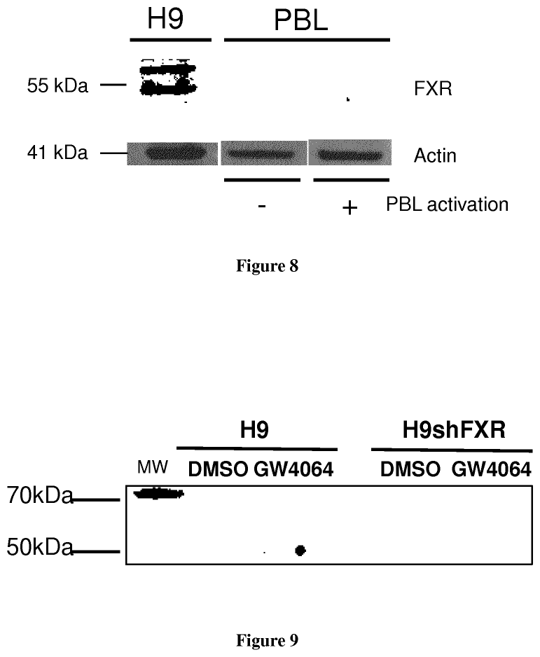

FXR is highly expressed in the liver, intestine and adrenal gland. It is also highly expressed in renal proximal tubule epithelial cells (7,8). On the opposite, expression of FXR in cells of the lymphoid lineage is less well established. FXR was detected in whole cell lysates by western blot analysis (FIG. 8) in H9 cell and in activated PBMC. No or a very faint band could be detected in non-activated PBMC. This difference suggests that PBMC activation induces FXR expression or that enrichment of CD4+T lymphocytes following activation allows the detection of FXR in these cells.

The effects of FXR agonist GW4064 on HIV-1 replication are abolished in the absence of FXR expression

Using shFXR lentiviral vector, an shFXR-H9 cell line was generated. FIG. 9 shows FXR was indeed silenced in this cell line. Interestingly, treatment of control H9 as well as that of shFXR-H9 cells further reduced FXR expression, suggesting FXR activation represses its own expression.

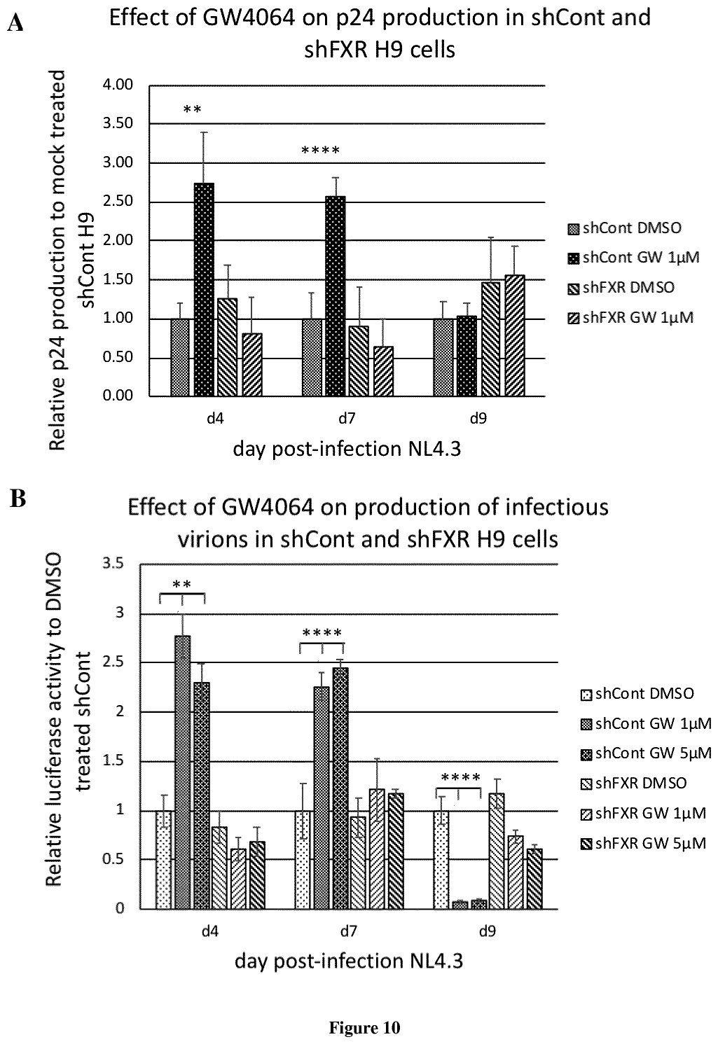

Treatment of shCont-H9 cells with HIV-1 NL4.3 with GW4064 at 1 μM again lead to an increased early production of p24 and infectious viral particles at day 4 and 7 post-infection compared to mock treated cells (FIGS. 10 A, B and C). This early boost of viral production was later followed by a strong decline of p24 concentrations and infectious titers at days 9. GW4064 treatment had no effect on viral production in shFXR-H9 cells, clearly indicating that the effects of this molecule are indeed dependent on FXR activation. Interestingly, GW4064 seemed to have already reached its maximum effect on the production of infectious particles at 1 μM.

FXR Agonists GW4064 or 6-ECDCA Contribute to Reactivate Latent Provirus

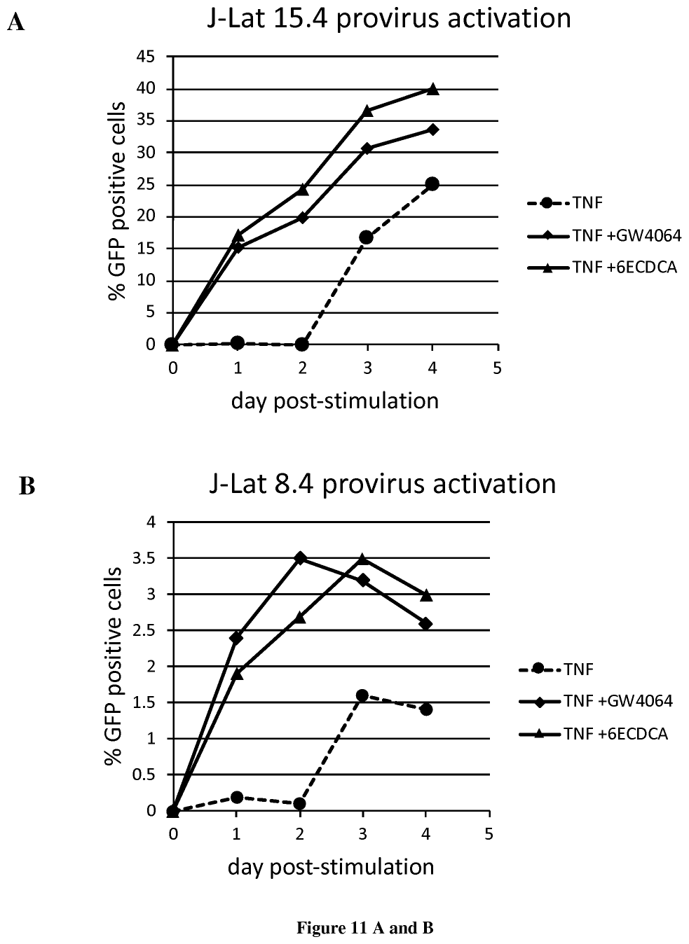

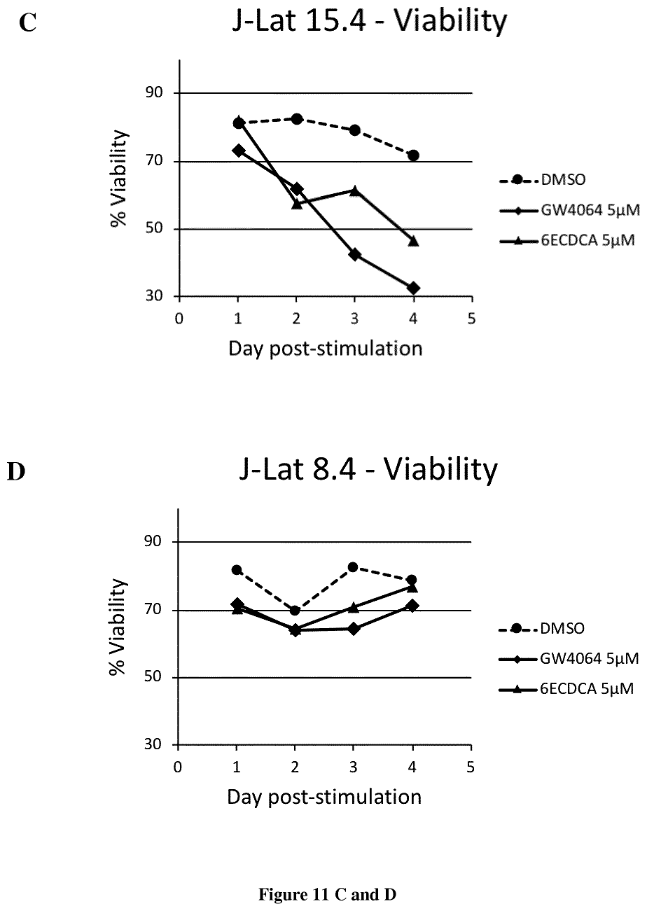

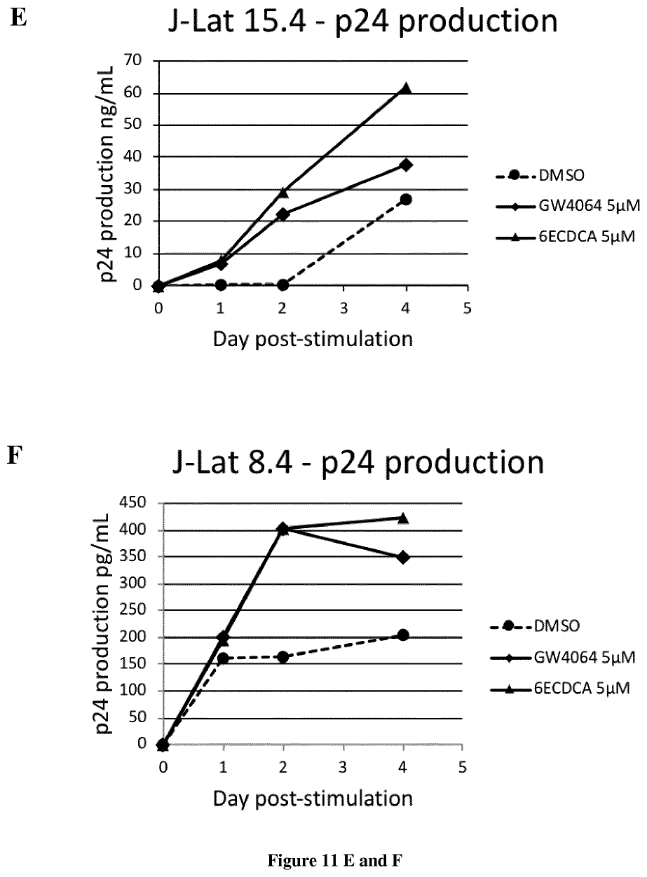

The effects of FXR agonists GW4064 and 6-ECDCA at 5 μM on latently infected cells were tested using Jurkat clones 8.4 and 15.4 described in (2). When cells were treated with TNFα at 0.5 ng/mL, percentages of GFP+ reactivated cells increased only at day 3 and 4 post-stimulation for both clones (FIGS. 11 A and B). However, reactivation reached higher percentages of 15.4 cells than 8.4. Co-stimulation with either one agonist induced a higher and earlier reactivation already detectable at day 1 post-stimulation. Reactivation was associated with increased cell death rates for clone 15.4 (FIG. 11 C). This effect could hardly be detected with clone 8.4 (FIG. 11 D), likely because the fraction of reactivated cells was too small to make this effect detectable, even if fractions of viable cells were consistently lightly lower in co-treated conditions than in TNFα treated cells. Production of p24 was also increased when cells were co-stimulated with FXR agonists (FIGS. 11 E and F). Again, clone 15.4 was more reactive than clone 8.4.

Effect of Treatment with FXR Ligands of Primary Human Renal Proximal Tubular Epithelial Cells (RPTEC) Infected with BKV

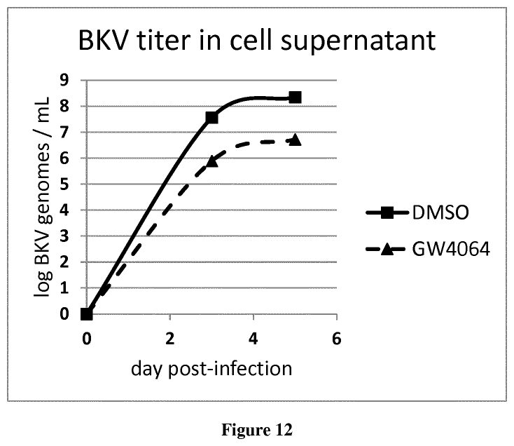

The effect of FXR modulation was then tested on replication of BKV, a member of the Polyomaviridae family in primary human renal proximal tubular epithelial cells (RPTEC) that express high level of FXR (data not shown). Results of the effect of the FXR agonist GW4064 after 3 and 5 days of treatment is shown on FIG. 12. It was observed a reduction of more than 1.5 Log 10 of the titer of viruses produced in the supernatant of GW4064 treated cells compared to mock treated cells. Cell counts at day 5 indicated no significant different cell numbers in non-infected cells whether they were treated or not (45200+/−10888 versus 59400+/−7660 respectively p>0.13). On the opposite, counts of infected cells showed a significant lower number of treated than untreated cells (44800+/−7989 versus 74800+/−13163 respectively, p=0.028) suggesting that FXR agonist treatment increased cell death rate in infected culture (not shown).

### CONCLUSIONS

Surprisingly we found that FXR agonists are active on virus replication in tissues that are not dedicated to bile salts metabolism and transport. This new finding clearly indicates that the nuclear receptor FXR has functions that largely expand over metabolism. Indeed, we found three major unexpected effects of FXR agonist treatment on viruses whose replication involves a viral DNA genome intermediate. First, FXR agonists, but not antagonist, substantially reduce the replication of two paragon viruses, which have DNA episomal intermediate of replication with or without viral genome integration into the host cell chromatin, and which rely on host machinery for viral mRNA transcription. Actually, with respect to HIV-1, the effect of FXR agonists is biphasic with a first initial and transient boost of virus production that is followed by a sharp and profound decline of viral replication. Second, another important and surprising finding is the high rate of infected cell death induced by FXR activation that participates to the antiviral activity. And third and again unexpectedly, FXR agonists reactivate silent retroviral integrated proviruses. Reactivation is then followed again by an increased cell death rate. The role of the engagement of FXR in the effects induced by FXR agonists is demonstrated by the lack of any effect in cells invalidated for FXR expression.

It is of prime importance that the effects are observed with two viruses with very dissimilar replication cycles but which have in common the presence of episomal forms of DNA whether or not these DNA genomes directly derived from the genome included in circulating virions or generated by reverse transcription from genomic RNA. Viral transcription thus is regulated from these DNA intermediates, which can be integrated or not in the host cell chromatin. Taking into account the effect observed on HBV replication, another virus that relies on an episomal intermediate of replication for its maintenance and transcription, these new findings indicate that FXR manipulation with ligands is interesting in treating infections with viruses sharing these traits, even in tissues not implicated in bile salts metabolism.

Important issues are also the reactivation of HIV-1 latent proviruses and the cell mortality induced by FXR agonist treatment. These findings are of special interest for HIV-1 infection for which the main therapeutic goal is no longer to develop new direct antiretroviral, many approved direct acting antiretrovirals are potent viral suppressors, but instead to clear the viral reservoir characterized by cells permanently carrying the viral genome. Favoring both latent provirus reactivation and the death of infected cells are thus important tools to decrease the reservoir of infected cells.

Other FXR ligands with poor “agonist” activity as measured by the state of the art assays may be as, or more, potent anti-viral than currently available FXR agonists. Assays based on the antiviral activity against HIV or other viruses with episome intermediate can be used for screening of FXR ligands with antiviral activity. Alternatively, agonists may induce the expression of some so far non-identified genes, under the control of FXR, which may participate to the clearance of extra-chromosomal DNA.

Indications of treatment with FXR agonists extend thus to most DNA viruses that infect human with the objectives to repress their replication and clear the infected cell reservoir.

### REFERENCES

Throughout this application, various references describe the state of the art to which this invention pertains. The disclosures of these references are hereby incorporated by reference into the present disclosure.
[1] Maloney P R, Parks D J, Haffner C D, Fivush A M, Chandra G, Plunket K D, et al. Identification of a chemical tool for the orphan nuclear receptor FXR. J Med Chem 2000; 43:2971-4.
[2] Kimpton J, Emerman M. Detection of replication-competent and pseudotyped human immunodeficiency virus with a sensitive cell line on the basis of activation of an integrated beta-galactosidase gene. J Virol 1992; 66:2232-9.
[3] Adachi A, Gendelman H E, Koenig S, Folks T, Willey R, Rabson A, et al. Production of acquired immunodeficiency syndrome-associated retrovirus in human and nonhuman cells transfected with an infectious molecular clone. J Virol 1986; 59:284-91.
[4] Sharma B N, Li R, Bernhoff E, Gutteberg T J, Rinaldo C H. Fluoroquinolones inhibit human polyomavirus BK (BKV) replication in primary human kidney cells. Antiviral Res 2011; 92:115-23. doi:10.1016/j.antiviral.2011.07.012.
[5] Lefebvre P, Cariou B, Lien F, Kuipers F, Staels B. Role of Bile Acids and Bile Acid Receptors in Metabolic Regulation. Physiol Rev 2009; 89:147-91. doi:10.1152/physrev.00010.2008.
[6] Mazuy C, Helleboid A, Staels B, Lefebvre P. Nuclear bile acid signaling through the farnesoid X receptor. Cell Mol Life Sci 2015; 72:1631-50. doi:10.1007/s00018-014-1805-y.
1. Kimpton J, Emerman M. Detection of replication-competent and pseudotyped human immunodeficiency virus with a sensitive cell line on the basis of activation of an integrated beta-galactosidase gene. J. Virol.1992; 66:2232-2239.
2. Jordan A, Bisgrove D, Verdin E. HIV reproducibly establishes a latent infection after acute infection of T cells in vitro. EMBO J. 2003; 22:1868-1877.
3. Adachi A, Gendelman H E, Koenig S, Folks T, Willey R, Rabson A, et al. Production of acquired immunodeficiency syndrome-associated retrovirus in human and nonhuman cells transfected with an infectious molecular clone. J. Virol. 1986; 59:284-291.
4. Sharma B N, Li R, Bernhoff E, Gutteberg T J, Rinaldo C H. Fluoroquinolones inhibit human polyomavirus BK (BKV) replication in primary human kidney cells. Antiviral Res. 2011; 92:115-123.
5. Maloney P R, Parks D J, Haffner C D, Fivush A M, Chandra G, Plunket K D, et al. Identification of a chemical tool for the orphan nuclear receptor FXR. J. Med. Chem. 2000; 43:2971-2974.
6. Pellicciari R, Fiorucci S, Camaioni E, Clerici C, Costantino G, Maloney P R, et al. 6α-Ethyl-Chenodeoxycholic Acid (6-ECDCA), a Potent and Selective FXR Agonist Endowed with Anticholestatic Activity. J. Med. Chem. 2002; 45:3569-3572.
7. Lefebvre P, Cariou B, Lien F, Kuipers F, Staels B. Role of Bile Acids and Bile Acid Receptors in Metabolic Regulation. Physiol Rev. 2009; 89:147-191.
8. Mazuy C, Helleboid A, Staels B, Lefebvre P. Nuclear bile acid signaling through the farnesoid X receptor. Cell. Mol. Life Sci. 2015; 72:1631-1650.

1. A method of reducing persistence and expression of an episomal virus in a subject in need thereof comprising administrating to the subject a therapeutically effective amount of a farnesoid X receptor (FXR) agonist.

2. The method of claim 1 wherein the subject is a human or a non human animal.

3. The method of claim 2 wherein the subject is a domestic animal or a farm animal.

4. The method of claim 1 wherein the episomal virus is selected from the group consisting of Adenoviridae, Retroviridae, Herpesviridae, Papovaviridae, Papillomaviridae, Polyomaviridae, and Parvoririnae families.

5. The method of claim 1 wherein the episomal virus is an adenovirus, a herpesvirus, a papillomavirus, a polyomavirus, a parvovirus or a retrovirus.

6. The method of claim 1 wherein the episomal virus is selected from the group consisting of BKV, CMV, EBV, HHV8 and HIV.

7. The method of claim 1 wherein the subject is immunocompromised.

8. The method of claim 1 wherein the subject has a cancer and is treated with a cytoablative therapy such as chemotherapy or radiotherapy.

9. The method of claim 1 wherein the subject is a transplant subject who is treated with an immunosuppressive agent.

10. A method of eradicating an HIV reservoir in a subject in need thereof after highly active antiretroviral treatment, comprising
administering to the subject a therapeutically effective amount of a FXR agonist.

11. The method of claim 3 wherein the domestic animal is a cat or dog.

12. The method of claim 3 wherein the farm animal is a horse, cow, pig or chicken.

13. The method of claim 7, wherein the immunocompromised subject is an elderly patient, an AIDS patients, a patient on a chronic immunosuppressive treatment regimen, a patient with cancer or a patient with an autoimmune condition

14. The method of claim 13, wherein the patient on a chronic immunosuppressive treatment regimen is an organ transplant recipient.

15. The method of claim 13, wherein the cancer is Hodgkin's disease or lymphoma.

16. The method of claim 13, wherein the patient with an autoimmune condition is being treated with mycophenolate mofetil, natalizumab, rituximab, or efalizumab.

17. The method of claim 13, wherein the autoimmune condition is multiple sclerosis (MS), rheumatoid arthritis (RA), or systemic lupus erythematosis (SLE).

18. The method of claim 8, wherein the cytoablative therapy is chemotherapy or radiotherapy.
